(19) United States
(12) Patent Application Publication
(10) Pub. No.: US 2014/0272936 A1
(43) Pub. Date: Sep. 18, 2014

(54) ADULTERATION TESTING OF HUMAN MILK

(71) Applicant: Prolacta Bioscience, City of Industry, CA (US)

(72) Inventors: Scott EAKER, City of Industry, CA (US); Mark A. Wisniewski, City of Industry, CA (US)

(73) Assignee: Prolacta Bioscience, City of Industry, CA (US)

(21) Appl. No.: 14/198,452

(22) Filed: Mar. 5, 2014

Related U.S. Application Data
(60) Provisional application No. 61/779,774, filed on Mar. 13, 2013.

(51) Int. Cl. G01N 33/53; G01N 33/04

(52) U.S. Cl. CPC G01N 33/5308; G01N 33/04
USPC 435/5; 436/23; 435/7.92; 436/501; 435/34

(57) ABSTRACT

The present invention provides a method for screening human milk for an adulterant, e.g., non-human milk and infant formula, as well as methods of making human milk compositions free of an adulterant, e.g., human milk fortifiers and standardized human milk formulations.

### CROSS-REFERENCE TO RELATED APPLICATIONS

This application claims priority to U.S. Provisional Application No. 61/779,774 filed Mar. 13, 2013, the contents of which are herein incorporated by reference in its entirety.

### FIELD OF THE INVENTION

The disclosure relates to a method for screening human milk for adulterants, e.g., non-human milk and infant formula, and methods of making human milk compositions free of adulterants.

### BACKGROUND OF THE INVENTION

Human milk is generally the food of choice for preterm and term infants because of its nutritional composition and immunologic benefits. The source of human milk can be, e.g., a donor or the infant's mother. Donors may or may not be compensated, e.g., monetarily, for their donations. Human breast milk donors tend to pump their milk for donation at home or other locations convenient to them and then often store the breast milk in their freezers until they have accumulated enough to bring or send to the donation center. Thus, in the absence of direct supervision of the donations, questions may arise as to the composition or purity of the donation. Specifically, donors who are being compensated for their donation may be motivated to increase the volume of their donation by adding non-human milk. In order to prevent the use of human milk that has been adulterated, e.g., with non-human milk or infant formula, there is a need for a reliable and sensitive method for detecting the presence of adulterants in human milk is featured herein.

### SUMMARY OF THE INVENTION

The methods featured herein relate to screening or testing human milk samples for any number of adulterants and producing human milk compositions free of an adulterant. In one aspect, the invention provides methods for screening human milk samples to confirm that the milk has not been mixed with non-human milk or infant formula.

In one aspect, the disclosure features a method for screening human milk for an adulterant comprising obtaining a sample of the human milk and screening the human milk sample for one or more adulterants, wherein a positive result indicates the human milk is adulterated and a negative result indicates the human milk is free of the one or more adulterants. In one embodiment, the adulterant is a non-human milk or an infant formula. In a related embodiment, the non-human milk is cow milk, goat milk, or soy milk. In another embodiment, the infant formula is cow formula (e.g., a cow-based infant formula) or soy formula (e.g., a soy-based infant formula).

In one embodiment, the screening step comprises an ELISA. The ELISA may be manual or automated. In one embodiment, the sample is not extracted prior to screening.

In certain embodiments, the human milk is pooled from two or more individuals. In a particular embodiment, the human milk is pooled from ten or more individuals.

In one embodiment, the human milk is frozen prior to screening. In another embodiment the human milk is not frozen prior to screening.

In another aspect, the disclosure provides a method for obtaining a pool of human milk free of an adulterant comprising obtaining human milk from two or more individuals; mixing the human milk from the two or more individuals, thereby providing a pool of human milk; obtaining a sample from the pool of human milk; screening the sample for one or more adulterants, wherein a positive result indicates the pool of human milk is adulterated and a negative result indicates the pool of human milk is free of the one or more adulterants; and selecting the pool of human milk with the negative result, thereby obtaining a pool of human milk free of an adulterant.

In one embodiment, the adulterant is a non-human milk or an infant formula. In a related embodiment, the non-human milk is cow milk, goat milk, or soy milk. In another embodiment, the infant formula is cow formula or soy formula.

In a particular embodiment, the screening step comprises an ELISA. The ELISA may be manual or automated.

In one embodiment, the sensitivity of the screening is more than about 80%, or more than about 85% or more than about 90% or more than about 95% or more than about 99%. In a further embodiment, the specificity of the screening is more than about 80% or more than about 90% or more than about 95% or more than about 99%, for example 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%.

In one embodiment, the human milk that is screened for the presence of an adulterant is also screened for the presence of one or more pathogens and/or drugs. In one embodiment, the human milk is screened for B. cereus, HIV-1, HBV and/or HCV. In a particular embodiment, the milk is screened for B. cereus, HIV-1, HBV and HCV. In one embodiment the milk is screened for amphetamine, benzodiazepine, cocaine, marijuana, methamphetamine, opiates, synthetic opioids (e.g. oxycodone/oxymorphone), and/or nicotine. In a further embodiment, the milk is screened for amphetamine, benzodiazepine, cocaine, marijuana, methamphetamine, opiates, synthetic opioids (e.g. oxycodone/oxymorphone) and nicotine.

In one embodiment, the pool of human milk is from ten or more individuals. In another embodiment, the human milk is frozen prior to screening.

Another aspect, the disclosure provides a method of making a fortified pool of human milk free of an adulterant comprising obtaining human milk from 2 or more individuals; mixing the human milk from the two or more individuals, thereby providing a pool of human milk; obtaining a sample from the pool of human milk; screening the sample for one or more adulterants, wherein a positive result indicates the pool of human milk is adulterated and a negative result indicates the pool of human milk is free of the one or more adulterants; selecting the pool of human milk with the negative result, thereby obtaining a pool of human milk free of an adulterant; and processing the pool of human milk free of an adulterant to obtain a fortified pool of human milk free of an adulterant, wherein the processing comprises: screening the pool of human milk free of an adulterant for the presence of pathogens, drugs and contaminants; conducting a nutritional analysis on the pool of human milk free of an adulterant; conducting a fortification of the pool of human milk free of an adulterant thereby obtaining a fortified pool of human milk free of an adulterant; and pasteurizing the fortified pool of human milk free of an adulterant.

In one embodiment, the fortified pool of human milk free of an adulterant comprises a human protein constituent of 35-85 mg/mL, a human fat constituent of 60-110 mg/mL, and a human carbohydrate constituent of 60-140 mg/mL. In another embodiment, the fortified pool of human milk free of an adulterant comprises a human protein constituent of 9-20 mg/mL, a human fat constituent of 35-55 mg/mL, and a human carbohydrate constituent of 70-120 mg/mL.

In another aspect, the disclosure provides a method of making a standardized human milk formulation free of an adulterant comprising obtaining human milk from 2 or more individuals; mixing the human milk from the two or more individuals, thereby providing a pool of human milk; obtaining a sample from the pool of human milk; screening the sample for one or more adulterants, wherein a positive result indicates the pool of human milk is adulterated and a negative result indicates the pool of human milk is free of the one or more adulterants; selecting the pool of human milk with the negative result, thereby obtaining a pool of human milk free of an adulterant; and processing the pool of human milk free of an adulterant to obtain a standardized human milk formulation free of an adulterant, wherein the processing comprises: screening the pool of human milk free of an adulterant for the presence of pathogens, drugs and contaminants; conducting a nutritional analysis on the pool of human milk free of an adulterant; conducting nutrient standardization of the pool of human milk free of an adulterant thereby obtaining a standardized human milk formulation free of an adulterant; and pasteurizing the fortified pool of human milk free of an adulterant.

In one embodiment, the standardized human milk formulation free of an adulterant comprises a human protein constituent of about 15-35 mg/mL or about 20-30 mg/mL or about 25-35 mg/mL and a human fat constituent of about 30-65 mg/mL or about 40-55 mg/mL or about 50-65 mg/mL.

In another aspect, the disclosure provides a method of making a human milk derived cream formulation free of an adulterant comprising obtaining human milk from 2 or more individuals; mixing the human milk from the two or more individuals, thereby providing a pool of human milk; obtaining a sample from the pool of human milk; screening the sample for one or more adulterants, wherein a positive result indicates the pool of human milk is adulterated and a negative result indicates the pool of human milk is free of the one or more adulterants; selecting the pool of human milk with the negative result, thereby obtaining a pool of human milk free of an adulterant; and processing the pool of human milk free of an adulterant to obtain a human milk derived cream formulation free of an adulterant, wherein the processing comprises: screening the pool of human milk free of an adulterant for the presence of pathogens, drugs and contaminants; separating the skim from the cream and standardizing the cream fraction thereby obtaining a human milk derived cream formulation free of an adulterant; and pasteurizing the human milk derived cream formulation free of an adulterant.

In one embodiment, the human milk derived cream formulation comprises from about 1.5 kcal/mL to about 3.5 kcal/mL, for example about 2.0 kcal/mL or about 2.5 kcal/mL or about 3.0 kcal/mL or about 3.0 kcal/mL. In one embodiment, the human milk derived cream formulation comprises from about 15% to about 35% fat, for example 20% fat, 25% fat, or 30% fat.

In another aspect, the disclosure provides a method of making a human milk derived oligosaccharide formulation free of an adulterant comprising obtaining human milk from 2 or more individuals; mixing the human milk from the two or more individuals, thereby providing a pool of human milk; obtaining a sample from the pool of human milk; screening the sample for one or more adulterants, wherein a positive result indicates the pool of human milk is adulterated and a negative result indicates the pool of human milk is free of the one or more adulterants; selecting the pool of human milk with the negative result, thereby obtaining a pool of human milk free of an adulterant; and processing the pool of human milk free of an adulterant to obtain a human milk derived oligosaccharide formulation free of an adulterant, wherein the processing comprises: screening the pool of human milk free of an adulterant for the presence of pathogens, drugs and contaminants; separating the skim from the cream, further filtering the skim portion, for example by ultrafiltration, to obtain a human milk permeate, and processing the human milk permeate, for example by concentration (i.e. reverse osmosis) to obtain a human milk derived oligosaccharide formulation free of an adulterant.

Another aspect of the disclosure features a method of making a processed human milk composition free of an adulterant comprising obtaining human milk from 2 or more individuals; mixing the human milk from the two or more individuals, thereby providing a pool of human milk; obtaining a sample from the pool of human milk; screening the sample for one or more adulterants, wherein a positive result indicates the pool of human milk is adulterated and a negative result indicates the pool of human milk is free of the one or more adulterants; selecting the pool of human milk with the negative result, thereby obtaining a pool of human milk free of an adulterant; and further processing the pool of human milk free of an adulterant to obtain a processed human milk composition free of an adulterant, wherein the processing comprises: filtering the pool of human milk free of an adulterant through a filter of about 100-400 microns; heat treating the pool of human milk free of an adulterant at about 58-65° C. for about 20-40 minutes; separating the pool of human milk free of an adulterant into a skim portion and a fat portion; filtering the skim portion through one or more skim filters to obtain a permeate portion and a protein rich skim portion; heating the fat portion to a temperature of about 90-120° C. for about one hour sufficient to reduce the bioburden of the fat portion; and mixing a fraction of the processed fat portion with the protein rich skim portion to obtain a processed human milk composition free of an adulterant.

In one embodiment, the processed human milk composition free of an adulterant comprises a human protein constituent of 35-85 mg/mL, a human fat constituent of 60-110 mg/mL, and a human carbohydrate constituent of 60-140 mg/mL. In another embodiment, the processed human milk composition free of an adulterant comprises a human protein constituent of 11-20 mg/mL, a human fat constituent of 35-55 mg/mL, and a human carbohydrate constituent of 70-120 mg/mL.

In on aspect, the invention provides for methods of qualifying donors based on the purity of their donated milk samples. In one embodiment, a donor is disqualified if her donated human milk sample contains an adulterant. In another embodiment, the donor may be qualified if her donated milk sample does not contain an adulterant. In one embodiment, the adulterant is a non-human milk or infant formula. In one embodiment, the non-human milk is cows milk, goat milk or soy milk. In another embodiment the adulterant is an infant formula. In one embodiment, the infant formula is a soy-based formula. In another embodiment, the infant formula is a cow's milk based formula. In one embodiment, the donated milk sample is tested for the presence of an adulterant by ELISA. In one embodiment, the ELISA is automated.

### BRIEF DESCRIPTION OF THE DRAWINGS

FIG. 1 is a bar graph that shows the effect of storage duration, storage temperature and number of freeze-thaw cycles on detection with the Veratox® Total Milk Allergen assay. “COCAL” refers to the cutoff calibrator prepared the day of the assay.

FIG. 2 is a bar graph that shows the effect of storage duration, storage temperature and number of freeze-thaw cycles on detection with the Veratox® Soy Allergen assay. “COCAL” refers to the cutoff calibrator prepared the day of the assay.

### DETAILED DESCRIPTION

This disclosure features a method of screening human milk for an adulterant, e.g., non-human milk and infant formula, and methods of making human milk compositions free of an adulterant. Screening or testing a human milk donation for adulterants ensures the donation contains only human milk. The donated milk is most often donated without supervision of personnel of the organization that will be receiving the milk, e.g., a milk bank center. Particularly when donors are compensated for milk donations, it is desirable to confirm that the volume of the donation has not been increased by the addition of non-human milk in order to avoid problems associated with feeding non-human milk to infants. For example, infants receiving the donation or a product made using the donation may have an allergy or sensitivity to non-human milk. Alternatively, the infant receiving the donation or a product made using the donation may be a very low birth weight infant, and therefore to decrease the incidence and/or severity of necrotizing Enterocolitis, will have a need to receive an exclusively human milk diet (See Sullivan, et al. (2010) J. Ped. 156(4):562-567) Additionally, it is desirable to be able to detect adulterants from a small volume of the donation so as to not waste the donation on testing. Furthermore, it is desirable to be able to test pooled samples of milk, for example, up to 10 donors in one pool, and therefore a sensitive test is required to be able to detect diluted levels of adulterant that may be present in one donation but not in other donations in the pool. While methods exist in the art to test for the presence or absence of non-human milk allergens (for example, cow's milk allergens, or soy milk allergens, etc) these methods have not been employed to test other milks for the presence or absence of these allergens. Testing human milk for the presence or absence of other species (animal, plant or synthetic) is particularly challenging for a couple of reasons. First, it is difficult to detect only non-human milk proteins within a human milk solution given the complexity of the human milk solution and possible cross-reactivities between proteins of different species. Secondly, lactating females who donate milk often consume milk from other species as a part of their diet and some of the constituents of these milks may pass into their breast milk that is tested. Therefore, it is important to be able to detect the difference between adulteration (e.g. purposefully diluting the human milk donation with other species milk) and the presence of other species milk constituents in the donated milk sample due simply to consumption of these other species milks by the mother. Therefore, while methods are known in the art to test other food items for these non-human milks, testing a sample of human milk for these has not been heretofore reported due to the complexities of the starting material and the need for assay customization and optimization.

As used herein, the term “adulterant” refers to any non-human milk found in human milk. The addition of adulterants to human milk is referred to as “adulteration”. Examples of adulterants include milk from non-human species (e.g., cow milk, goat milk, etc.), milk-like products from plants (e.g., soy milk) and infant formula.

As used herein, the term “contaminant” refers to the inclusion of unwanted substances in human milk. While an adulterant is a “contaminant” generally the use of the term “contaminant” as used herein generally refers to other substances such as drugs, environmental pollutants and/or bacteria and viruses. The inclusion of contaminants to human milk is referred to as “contamination.” The inclusion of contaminants may be due to any reason including but not limited to accident, negligence or intent.

The terms “human milk”, “breast milk”, “donor milk”, and “mammary fluid” are used interchangeably and refer to milk from a human.

The term “infant formula” herein refers to commercially available infant nutritional products often sold as an alternative or additive to human milk based nutrition. Such formulas can contain milks from other species, i.e. cow or plant-based milk (i.e. soy) or maybe “synthetic” or produced by the hands of man. Such “synthetic milks” contain all of the constituents of human milk but are derived from non-human sources and/or are not purified directly from another animal or plant.

The terms “donor” and “individual” are used interchangeably and refer to a woman who supplies or provides a volume of her milk, regardless of whether or not she is compensated, e.g., monetarily, for the milk.

The terms “premature”, “preterm” and “low-birth-weight (LBW)” infants are used interchangeably and refer to infants born less than 37 weeks gestational age and/or with birth weights less than 2500 gm.

By “whole milk” is meant milk from which no fat has been removed.

By “bioburden” is meant microbiological contaminants and pathogens (generally living) that can be present in milk, e.g., viruses, bacteria, mold, fungus and the like.

All patents, patent applications, and references cited herein are incorporated in their entireties by reference.

Obtaining Human Milk from Qualified and Selected Donors

The methods of the present disclosure utilize human milk. Various techniques are used to identify and qualify suitable donors. A potential donor must obtain a release from her physician and her child's pediatrician as part of the qualification process. This helps to insure, inter alia, that the donor is not chronically ill and that her child will not suffer as a result of the donation(s). Methods and systems for qualifying and monitoring milk collection and distribution are described, e.g., in U.S. patent application Ser. No. 11/526,127 (U.S. 2007/0098863), which is incorporated herein by reference in its entirety. The current invention describes an additional qualification screening. In particular, the method of the present invention includes qualifying donors based on the presence or absence of one or more adulterants in their donated milk samples. In a particular embodiment, donors are disqualified if their donated milk sample comprises an adulterant.

Donors may be periodically requalified. For example, a donor is required to undergo screening by the protocol used in their initial qualification every four months, if the donor wishes to continue to donate. A donor who does not requalify or fails qualification is deferred until such time as they do, or permanently deferred if warranted by the results of requalification screening. In the event of the latter situation, all remaining milk provided by that donor is removed from inventory and destroyed.

A qualified donor may donate at a designated facility (e.g., a milk bank office) or, typically, expresses milk at home. The qualified donor can be provided with supplies by a milk bank or directly from a milk processor (the milk bank and processor may be the same or different entities) to take home. The supplies will typically comprise a computer readable code (e.g., a barcode-label) on containers and may further include a breast pump. The containers may also include a programmable chip that records and stores data related to, e.g., temperature variations, handling conditions, contents, origin of contents, date shipped, date received, lot numbers and/or any other information required for quality control, regulatory or other reasons. The donor may then pump and freeze the milk at home at a temperature of about −20° C. or colder. The donor milk is accepted, provided that the donor is a qualified donor; if such results are satisfactory, an appointment is made for the donor to drop off the milk at the center, or to have it collected from home. A donor can also ship the milk directly to the milk bank center or milk processor in insulated containers provided by the milk bank or milk processor. The milk and container are examined for their condition and the barcode information checked against the database. If satisfactory, the units are placed in the donor milk center or processing center freezer (−20° C. or colder) until ready for further testing and processing.

Screening for Contaminants

Generally, the donor screening process includes both interviews and biological sample processing. Any blood sample found positive for, e.g., viral contamination, on screening removes the donor from the qualification process.

Once a donor qualifies and begins sending milk, milk from each of her shipments is tested for, e.g., B. cereus, HIV-1, HBV, HCV and drugs of abuse (e.g., cotinine, cocaine, opiates, synthetic opioids (e.g. oxycodone/oxymorphone), nicotine, methamphetamines, benzodiazepine, amphetamines, and THC including their principle metabolites). The milk may be genetically screened, e.g., by polymerase chain reaction (PCR), to identify any contaminants, e.g., viral, e.g., HIV-1, HBV, and/or HCV. Any positive finding results in the deferral of the donor and destruction of all previously-collected milk or the removal of the donation to be used only for research purposes.

Testing Donor Identity

Because in some embodiments of the present methods the milk is expressed by the donor at, e.g., her home and not collected at the milk banking facility, each donor's milk is sampled for genetic markers, e.g., DNA markers, to guarantee that the milk is truly from the registered donor. Such subject identification techniques are known in the art (see, e.g., International Application Serial No. PCT/US2006/36827, which is incorporated herein by reference in its entirety). The milk may be stored (e.g., at −20° C. or colder) and quarantined until the test results are received.

For example, the methods featured herein may include a step for obtaining a biological reference sample from a potential human breast milk donor. Such sample may be obtained by methods known in the art such as, but not limited to, a cheek swab sample of cells, or a drawn blood sample, milk, saliva, hair roots, or other convenient tissue. Samples of reference donor nucleic acids (e.g., genomic DNA) can be isolated from any convenient biological sample including, but not limited to, milk, saliva, buccal cells, hair roots, blood, and any other suitable cell or tissue sample with intact interphase nuclei or metaphase cells. The sample is labeled with a unique reference number. The sample can be analyzed at or around the time of obtaining the sample for one or more markers that can identify the potential donor. Results of the analysis can be stored, e.g., on a computer-readable medium. Alternatively, or in addition, the sample can be stored and analyzed for identifying markers at a later time.

It is contemplated that the biological reference sample may be DNA typed by methods known in the art such as STR analysis of STR loci, HLA analysis of HLA loci or multiple gene analysis of individual genes/alleles. The DNA-type profile of the reference sample is recorded and stored, e.g., on a computer-readable medium.

It is further contemplated that the biological reference sample may be tested for self-antigens using antibodies known in the art or other methods to determine a self-antigen profile. The antigen (or another peptide) profile can be recorded and stored, e.g., on a computer-readable medium.

A test sample of human milk is taken for identification of one or more identity markers. The sample of the donated human milk is analyzed for the same marker or markers as the donor's reference sample. The marker profiles of the reference biological sample and of the donated milk are compared. The match between the markers (and lack of any additional unmatched markers) would indicate that the donated milk comes from the same individual as the one who donated the reference sample. Lack of a match (or presence of additional unmatched markers) would indicate that the donated milk either comes from a non-tested donor or has been contaminated with fluid from a non-tested donor.

The donated human milk sample and the donated reference biological sample can be tested for more than one marker. For example, each sample can be tested for multiple DNA markers and/or peptide markers. Both samples, however, need to be tested for at least some of the same markers in order to compare the markers from each sample.

Thus, the reference sample and the donated human milk sample may be tested for the presence of differing identity marker profiles. If there are no identity marker profiles other than the identity marker profile from the expected subject, it generally indicates that there was no fluid (e.g., milk) from other humans or animals contaminating the donated human milk. If there are signals other than the expected signal for that subject, the results are indicative of contamination. Such contamination will result in the milk failing the testing.

The testing of the reference sample and of the donated human milk can be carried out at the donation facility and/or milk processing facility. The results of the reference sample tests can be stored and compared against any future donations by the same donor.

Throughout the processes described herein, any non-complying milk specimens are discarded, and the donor is disqualified. Access to all confidential information about the donor, including blood test data, is carefully controlled and meets Health Insurance Portability and Accountability Act (HIPAA) requirements.

### Screening Human Milk for Adulterants

As described herein, according to the present invention human milk is screened for one or more adulterants. The human milk may be provided by a donor that is compensated, e.g., monetarily, for the donation. In other instances, the donor is not compensated for the milk donation. A positive result indicates that the screening detected an adulterant in the human milk sample. In contrast, a negative result indicates that the human milk is free of the adulterant. Human milk that has been determined to be free of an adulterant, or was found to be negative for the adulterant, is selected and may be stored and/or further processed. Human milk that contains an adulterant will be discarded and the donor may be disqualified. For example, if an adulterant is found in two or more human milk samples from the same donor, the donor is disqualified. Surprisingly, the methods of the present invention reliably and reproducibly are able to detect adulterants in human milk directly without the need for time consuming and costly extractions. The methods are sensitive enough to detect even low levels of adulteration, but are specific enough to not cross react with human milk proteins or detect constituents in human breast milk derived from the consumption of the lactating donor of the particular adulterant.

Obtaining a Sample

Methods of obtaining a sample of frozen human milk include a stainless steel boring tool used to drill a core the entire length of the container. Alternatively, a sample may be scraped from the surface of the frozen human milk. The container may contain a separate portion for collection of a sample of the human milk, and this portion may be removed as the sample for testing. Where the human milk is in liquid form it is contemplated that the method for obtaining the test sample will be by pipette or other means. The container may include a one-way valve that allows for the release of a small amount of the human milk into a test vial while preventing contamination of the milk by pathogens.

If the sample is frozen, chunks of frozen human milk may be thawed using a slow, continuous heat and a mild churning action.

Adulterants

Adulterants include any non-human milk fluid or filler that is added to a human milk donation, thereby causing the donation to no longer be unadulterated, pure human milk. Particular adulterants to be screened for include non-human milk and infant formula. As used herein, “non-human milk” refers to both animal-, plant- and synthetically-derived milks. Examples of non-human animal milk include, but are not limited to, buffalo milk, camel milk, cow milk, donkey milk, goat milk, horse milk, reindeer milk, sheep milk, and yak milk. Examples of non-human plant-derived milk include, but are not limited to, almond milk, coconut milk, hemp milk, oat milk, rice milk, and soy milk. Examples of infant formula include, cow milk formula, soy formula, hydrolysate formula (e.g., partially hydrolyzed formula or extensively hydrolyzed formula), and amino acid or elemental formula. Cow milk formula may also be referred to as dairy-based formula. In particular embodiments, the adulterants that are screened for include cow milk, cow milk formula, goat milk, soy milk, and soy formula.

Screening Assays

According to the present invention, methods known in the art may be adapted to detect non-human milk proteins, e.g., cow milk and soy proteins, in a human milk sample. In particular, immunoassays that utilize antibodies specific for a protein found in an adulterant that is not found in human milk can be used to detect the presence of the protein in a human milk sample. For example, an enzyme-linked immunosorbent assay (ELISA), such as a sandwich ELISA, may be used to detect the presence of an adulterant in a human milk sample. An ELISA may be performed manually or be automated. Another common protein detection assay is a western blot, or immunoblot. Flow cytometry is another immunoassay technique that may be used to detect an adulterant in a human milk sample. ELISA, western blot, and flow cytometry protocols are well known in the art and related kits are commercially available. The use of commercially available ELISA kits adapted to be effective in detecting very low levels of cow milk, cow formula, goat milk, soy milk, and soy formula in human milk is demonstrated with sensitivity and specificity of over 95% in the Examples. Another useful method to detect adulterants in human milk is infrared spectroscopy and in particular mid-range Fourier transform infrared spectrometry (FTIR).

The human milk may be pooled prior to screening. In one embodiment, the human milk is pooled from more than one donation from the same individual. In another embodiment, the human milk is pooled from two or more, three or more, four or more, five or more, six or more, seven or more, eight or more, nine or more, or ten or more individuals. In a particular embodiment, the human milk is pooled from ten or more individuals. The human milk may be pooled prior to obtaining a sample by mixing human milk from two or more individuals. Alternatively, human milk samples may be pooled after they have been obtained, thereby keeping the remainder of each donation separate.

The screening step will yield a positive result if the adulterant is present in the human milk sample at about 20% or more, about 15% or more, about 10% or more, about 5% or more, about 4% or more, about 3% or more, about 2% or more, about 1% or more, or about 0.5% or more of the total volume of the milk donation.

The screening of the donated human milk for one or more adulterants can be carried out at the donation facility and/or milk processing facility.

### Processing Human Milk Free of Adulterants

The human milk screened by the methods featured herein can be processed for further use. The donation facility and milk processing facility can be the same or different facility. The donated milk that is free of an adulterant can be processed, e.g., to obtain human milk fortifiers, standardized human milk formulations, and/or human lipid compositions. Screening the donated human milk for adulterants ensures safety of the human milk and any products derived from such milk.

Processing of human milk to obtain human milk fortifiers (e.g., PROLACTPLUS™ Human Milk Fortifiers, e.g., PROLACT+4™, PROLACT+6™, PROLACT+8™, and/or PROLACT+10™, which are produced from human milk and contain various concentrations of nutritional components) and the compositions of the fortifiers are described in U.S. patent application Ser. No. 11/947,580, filed on Nov. 29, 2007, (U.S. Pat. No. 8,545,920) the contents of which are incorporated herein in their entirety. These fortifiers can be added to the milk of a nursing mother to provide an optimal nutritional content of the milk for, e.g., a preterm infant. Depending on the content of mother's own milk, various concentrations of the fortifiers can be added to mother's milk.

Methods of obtaining standardized human milk formulations (exemplified by Prolact HM™, Prolact RTF24™, Prolact RTF26™ and Prolact RTF 28™). These standardized human milk formulations can be used to feed, e.g., preterm infants, without mixing them with other fortifiers or milk. They provide a nutritional human-derived formulation and can substitute for mother's milk.

Compositions that include lipids from human milk (e.g., Prolact CR™) methods of obtaining such compositions, and methods of using such compositions to provide nutrition to patients are described in PCT Application PCT/US07/86973 filed on Dec. 10, 2007, and U.S. 61/779/781, filed Mar. 13, 2013, the contents of both of which are incorporated herein in their entireties.

Compositions that include human milk oligosaccharides (HMOs) from human milk, methods of obtaining such compositions and methods of using such compositions are described in PCT/US2009/066430, filed on Dec. 2, 2009, the contents of which incorporated by reference herein in its entirety.

Methods of obtaining other nutritional compositions from human milk that can be used with the methods featured herein are discussed in U.S. patent application Ser. No. 11/012,611, filed on Dec. 14, 2004, and published as U.S. 2005/0100634 on May 12, 2005, the contents of which are incorporated herein in their entirety.

Processing of milk that has been screened for adulterants can be carried out with large volumes of human milk, e.g., about 75 liters/lot to about 8,000 liters/lot of starting material.

The methods featured herein can also be integrated with methods of facilitating collection and distribution of human milk over a computer network, e.g., as described in U.S. patent application Ser. No. 11/526,127, filed on Sep. 22, 2006, and published as U.S. 2007/0098863 on May 3, 2007; and in U.S. patent application Ser. No. 11/679,546, filed on Feb. 27, 2007, and published as U.S. 2007/0203802 on Aug. 30, 2007. The contents of both applications are incorporated herein in their entireties.

Methods of Obtaining Human Milk Fortifiers and Human Milk-Based Products Free of Adulterants

Human milk is carefully analyzed for both identification purposes, as described above, and to avoid contamination. The milk is screened, e.g., genetically screened, e.g., by polymerase chain reaction (PCR). Genetic screening is done to identify any contaminants, e.g., viral, e.g., HIV-1, HBV, and/or HCV. The milk then undergoes filtering, e.g., through about a 200 micron filter, and heat treatment. For example, the composition can be treated at about 63° C. or greater for about 30 minutes or more. Next, the milk is transferred to a separator, e.g., a centrifuge, to separate the cream (i.e., the fat portion) from the skim. The skim can be transferred into a second processing tank where it remains at about 2 to 8° C. until a filtration step. Optionally, the cream separated from the skim, can undergo separation again to yield more skim.

Following the separation of cream and skim, a desired amount of cream is added to the skim, and the composition undergoes further filtration, e.g., ultrafiltration. This process concentrates the nutrients in the skim milk by filtering out the water. The water obtained during the concentration is referred to as the permeate. Filters used during the ultrafiltration can be postwashed and the resulting solution added to the skim to maximize the amount of nutrients obtained, e.g., obtaining a protein concentration of about 7% to 7.2%. The skim is then blended with the cream and samples taken for analysis. At this point during the process, the composition generally contains: about 8.5% to 9.5% of fat; about 6.3% to 7.0% of protein; and about 8% to 10.5% of carbohydrates, e.g., lactose.

After the separation of cream and skim, the cream flows into a holding tank, e.g., a stainless steel container. The cream can be analyzed for its caloric, protein and fat content. When the nutritional content of cream is known, a portion of the cream can be added to the skim milk that has undergone filtration, e.g., ultrafiltration, to achieve the caloric, protein and fat content required for the specific product being made. Minerals can be added to the milk prior to pasteurization. The cream can also be heated to a temperature of about 90-120° C. for about one hour to reduce the bioburden of the cream portion.

At this point, in one embodiment, the processed composition can be frozen prior to the addition of minerals and thawed at a later point for further processing. Any extra cream that was not used can also be stored, e.g., frozen. Optionally, before the processed composition is frozen, samples are taken for mineral analysis. Once the mineral content of the processed milk is known, the composition can be thawed (if it was frozen) and a desired amount of minerals can be added to achieve target values.

After blending the skim with the cream and/or the optional freezing and/or mineral addition, the composition undergoes pasteurization. For example, the composition can be placed in a process tank that is connected to the high-temperature, short-time (HTST) pasteurizer via platinum-cured silicone tubing. After pasteurization, the milk can be collected into a second process tank and cooled. Other methods of pasteurization known in the art can be used. For example, in vat pasteurization the milk in the tank is heated to a minimum of 63° C. and held at that temperature for a minimum of thirty minutes. The air above the milk is steam heated to at least three degrees Celsius above the milk temperature. In one embodiment, the product temperature is about 66° C. or greater, the air temperature above the product is about 69° C. or greater, and the product is pasteurized for about 30 minutes or longer. In another embodiment, both HTST and vat pasteurization are performed.

The resulting fortifier composition is generally processed aseptically. After cooling to about 2 to 8° C., the product is filled into containers of desired volumes, and various samples of the fortifier are taken for nutritional and bioburden analysis. The nutritional analysis ensures proper content of the composition. A label that reflects the nutritional analysis is generated for each container. The bioburden analysis tests for presence of contaminants, e.g., total aerobic count, B. cereus, E. coli, Coliform, Pseudomonas, Salmonella, Staphylococcus, yeast, and/or mold. Bioburden testing can be genetic testing. The product is packaged and shipped once the analysis is complete and desired results are obtained.

In one embodiment, the resultant fortified pool of human milk free of an adulterant comprises 35-85 mg/mL human protein, 60-110 mg/mL human fat, and 60-140 mg/mL human carbohydrate. In another embodiment, the resultant fortified pool of human milk free of an adulterant comprises 9-20 mg/mL human protein, 35-55 mg/mL human fat, and 70-120 mg/mL human carbohydrate.

Methods of Obtaining Standardized Human Milk Formulations Free of Adulterants

Human milk free of adulterants is screened to ensure the identity of the donors and reduce the possibility of contamination. The human milk is pooled and further screened, e.g., genetically screened (e.g., by PCR). The screening can identify, e.g., viruses, e.g., HIV-1, HBV, and/or HCV. Milk that tests positive is discarded.

After the screening, the composition undergoes filtering. The milk is filtered through about a 200 micron screen and then ultrafiltered. The milk may also be heat treated, e.g., the composition can be treated at about 58-65° C. or greater for about 20-40 minutes or more.

During ultrafiltration, water is filtered out of the milk (and is referred to as permeate) and the filters are postwashed using the permeate. Post wash solution is added to the milk to recover any lost protein and increase the concentration of the protein to, e.g., about 1.2% to about 1.5%. Cream from another lot (e.g., excess cream from a previous fortifier lot) is added to increase the caloric content. At this stage of the process, the composition generally contains: about 3.5% to 5.5% of fat; about 1.1% to 1.3% of protein; and about 8% to 10.5% of carbohydrates, e.g., lactose. The composition can be frozen and thawed out for further processing later.

Optionally, if the human milk formulation is to be fortified with minerals, a mineral analysis of the composition is carried out after cream is added. Once the mineral content is known, a desired amount of minerals can be added to achieve target values.

Next, the composition is pasteurized. Pasteurization methods are known in the art. For example, the product can be pasteurized in a tank that is jacketed. Hot glycol can be use to heat up the tank. The product temperature can be about 63° C. or greater and the air temperature above the product about 66° C. or greater. The product is pasteurized for a minimum of about 30 minutes. Other pasteurizing techniques are known in the art.

After cooling to about 2 to 8° C., the product is filled into containers of desired volumes and various samples of the human milk formulation are taken for nutritional and bioburden analysis. The nutritional analysis ensures proper content of the composition. A label generated for each container reflects the nutritional analysis. The bioburden analysis tests for presence of contaminants, e.g., total aerobic count, B. cereus, E. coli, Coliform, Pseudomonas, Salmonella, Staphylococcus, yeast, and/or mold. The product is packaged and shipped once the analysis is complete and desired results are achieved.

In one embodiment, the resultant processed human milk composition free of an adulterant comprises 15-35 mg/mL human protein or 20-30 mg/mL of human protein or 25-35 mg/mL of human protein and 30-65 mg/mL human fat, or 40-55 mg/mL of human fat or 50-65 mg/mL of human fat.

### EXAMPLES

The following examples are intended to illustrate but not limit the disclosure.

### Example 1

### Detection of Adulterants in Human Milk

To prevent the use of human milk that has been adulterated with non-human milk or infant formula, an assay to detect the presence of adulterants that uses very little breast milk is needed. This study was performed in order to determine if commercially available ELISA kits can be used to detect the presence of cow milk, goat milk, dairy-based infant formula, soy milk, and soy-based infant formula in human breast milk.

Veratox® ELISA kits for the detection of milk proteins, casein and whey, and soy proteins in food products are commercially available from Neogen Corporation. The Total Milk Allergen kit was used to screen for the presence of cow milk, goat milk, and dairy-based infant formula in human milk, and the Soy Allergen kit was used to screen for the presence of soy milk and soy-based infant formula in human milk. The kits were validated to screen for adulteration of human breast milk at a 10% adulteration cutoff level. Goat milk was used as the calibrator for the Total Milk Allergen kit and soy formula was used as the calibrator for the Soy Allergen kit. These cutoff calibrators were prepared using 1 mL of human breast milk spiked at an adulteration level of 10%. The assays were performed both according to the manufacturer's recommended procedure and without the recommended extraction step.

Human breast milk was provided by ten donors (15607 (A.1), 15966 (A.2), 16226 (B), 16528 (C), 16580 (D), 17046 (E), 17076 (F), 17193 (G), 17363 (H), and 17617 (I)). The non-human milk and infant formula samples used were purchased from a grocery store. The following five milk and infant formula samples were used as adulterants: Cow Milk (Hiland Vitamin D milk; Grade A; pasteurized and homogenated), Goat Milk (Meyenberg Ultra Pasteurized Vitamin D milk), Cow Milk-based Formula (Similac Advance Infant Formula; Complete Nutrition), Soy Milk (8^{th} Continent Soy Milk, Original flavor), and Soy-based Formula (Similac Soy Infant Formula; Isomil).

A pool of human breast milk was prepared by mixing equal volumes from donors A.2 (#15966), B (#16226), and C (#16528). Five mL aliquots from this pool of human breast milk were adulterated with 0%, 5%, 10%, or 20% of Cow Milk, Goat Milk, Cow Formula, Soy Milk, and Soy Formula. Test samples adulterated with 1% of Goat Milk, Soy Milk, and Soy Formula were also generated and screened. After mixing, 1 mL of the 5 mL aliquot was transferred to each of two 50 mL of conical tubes, and one was labeled as “Extraction”. A volume of 25 mL of 60° C. extraction buffer (i.e., PBS) was added to each tube. The “Extraction” tube also received ⅕ of a scoop of extraction additive, and was incubated in a shaking water bath at 60° C. for 15 min per the manufacturer's recommended protocol. After all extracted and non-extracted tubes had cooled to room temperature; the samples were diluted 1:100 and assayed using the ELISA kits.

The highest standard in each kit (25 ppm soy or 25 ppm non-fat dry milk) was included in the assay as a positive control, and PBS and 100% human breast milk were included as negative controls. The positive controls provided a positive result, and examples of the OD resulting from the negative controls are provided in Table 1 below. The cutoff calibrators were analyzed in triplicate and all samples in duplicate.

Volumes used were 1 mL of sample from 1 donor+25 mL of extraction buffer (phosphate buffered saline, PBS).

For samples that were extracted, the extraction buffer (PBS) was heated to 60° C. per the manufacturer's protocol. Additive was added to this sample (⅕ of a scoop for a 1 mL sample), followed by the appropriate volume of extraction buffer (PBS). Samples were then incubated in a 60° C. water bath for 15 minutes, while being shaken at 150 rpm. Samples were cooled to room temperature, and in the case of the Veratox® Soy Allergen test, were centrifuged (14,000 rpm for 5 min)

Samples were diluted with PBS to the appropriate dilutions in order to fit into the standard curve of the kit (1:100), and were analyzed with the appropriate ELISA assay. Samples were washed using an automatic plate washer (ten times for the Veratox® Total Milk Allergen kit and five times for the Veratox® Soy Allergen kit). Optical densities (“OD”) were measured using an Epoch plate reader at 650 nm. An OD value at least one standard deviation above the negative control is considered a positive result.

The Veratox® Total Milk ELISA assay provided an overall recovery of 96.3% (SD: 8.3, % CV: 8.6) for Cow Milk, Goat Milk, Cow Formula, and blank human breast milk (Negative), as depicted in Table 1. Percent Recovery was calculated by dividing the observed amount by the expected amount based on the dilution of the adulterant and multiplying by 100. The absorbance values (OD) were similar whether using extraction or no extraction with the Veratox® Total Milk ELISA kit.

TABLE 1
Absorbance Values of Various Adulteration level Samples Obtained
from the Veratox ® Total Milk ELISA Assay Using Extraction Step
Versus No Extraction

| | | Extraction | No Extraction | Percent |
|---|---|---|---|---|
| Run | Sample | 1 mL + 25 mL (PBS) | | Recovery |
| 1 | NEG. OD | 0.174 | 0.157 | 90.2 |
| 1 | 1% Goat Milk | 0.246 | 0.280 | 113.8 |
| 1 | 5% Goat Milk | 0.513 | 0.489 | 95.3 |
| 2 | 5% Goat Milk | 0.410 | 0.364 | 88.8 |
| 1 | 5% Cow Milk | 1.438 | 1.605 | 111.6 |
| 2 | 5% Cow Milk | 1.309 | 1.182 | 90.3 |
| 1 | 5% Cow Formula | 1.139 | 1.184 | 104 |
| 2 | 5% Cow Formula | 0.865 | 0.785 | 90.8 |
| | | | AVG. | 96.8 |
| | | | STD. DEV. | 9.1 |
| | | | % CV | 9.4 |
| 1 | 10% Goat Milk | 0.610 | 0.607 | 99.4 |
| 1 | 10% Goat Milk | 0.719 | 0.660 | 91.8 |
| 1 | 10% Goat Milk | 0.757 | 0.689 | 91 |
| 2 | 10% Goat Milk | 0.792 | 0.669 | 84.5 |
| 1 | 10% Cow Milk | 1.825 | 1.828 | 100.2 |
| 2 | 10% Cow Milk | 2.075 | 1.971 | 95 |
| 1 | 10% Cow Formula | 1.489 | 1.350 | 90.7 |
| 2 | 10% Cow Formula | 1.473 | 1.543 | 104.8 |
| | | | AVG. | 94.7 |
| | | | STD. DEV. | 6.5 |
| | | | % CV | 6.9 |
| 1 | 20% Goat Milk | 0.993 | 0.831 | 83.7 |
| 2 | 20% Goat Milk | 0.867 | 0.845 | 97.5 |
| 1 | 20% Cow Milk | 2.164 | 2.231 | 103.1 |
| 1 | 20% Cow Formula | 1.722 | 1.708 | 99.2 |
| | | | AVG. | 95.9 |
| | | | STD. DEV. | 8.5 |
| | | | % CV | 8.8 |

The Veratox® Soy ELISA provided an overall recovery of 98.3% (SD: 8.3, % CV: 8.6) for Soy Milk and Soy Formula. Similar to the Veratox® Total Milk ELISA, the absorbance values (OD) were similar whether using extraction or no extraction with the Veratox® Soy ELISA.

TABLE 2
Absorbance Values of Various Adulteration level Samples Obtained
from the Veratox ® Soy ELISA Assay Using Extraction Step
Versus No Extraction

| | | Extraction | No Extraction | Percent |
|---|---|---|---|---|
| Run | Sample | 1 mL + 25 mL (PBS) | | Recovery |
| 2 | 1% Soy Formula | 0.093 | 0.096 | 103 |
| 2 | 1% Soy Milk | 0.116 | 0.119 | 103 |
| | | | AVG. | 102.9 |
| | | | STD. DEV. | 0.5 |
| | | | % CV | 0.4 |
| 2 | 5% Soy Formula | 0.206 | 0.198 | 96 |
| 1 | 5% Soy Milk | 0.336 | 0.332 | 101 |
| 2 | 5% Soy Milk | 0.358 | 0.319 | 89 |
| | | | AVG. | 95.5 |
| | | | STD. DEV. | 6.1 |
| | | | % CV | 6.4 |
| 1 | 10% Soy Formula | 0.383 | 0.38 | 102 |
| 2 | 10% Soy Formula | 0.376 | 0.364 | 97 |
| 2 | 10% Soy Formula | 0.392 | 0.375 | 96 |
| 2 | 10% Soy Milk | 0.567 | 0.565 | 100 |
| | | | AVG. | 97.4 |
| | | | STD. DEV. | 2.1 |
| | | | % CV | 2.1 |
| 2 | 20% Soy Milk | 0.982 | 0.856 | 87 |
| 2 | 20% Soy Formula | 0.645 | 0.626 | 97 |
| | | | AVG. | 92.1 |
| | | | STD. DEV. | 7.0 |
| | | | % CV | 7.6 |

This study demonstrated that cow milk, goat milk, cow milk-based infant formula, soy milk, and soy-based infant formula could be detected in human milk by ELISA. In addition, the extraction step could be eliminated in both the Veratox® Total Milk Allergen and Veratox® Soy Allergen kits without negatively affecting the assay results. Removing this step saves a considerable amount of time during sample preparation.

### Example 2

### Detection of Adulterants in Smaller Samples of Pooled Human Milk

This study was performed in order to determine if ten donors could be pooled per test sample for screening purposes, and if using a reduced sample volume of 100 μL per donor would produce similar results to those obtained using a sample volume of 1 mL.

Different donor volumes were compared to the results obtained in Example 1. For both kits samples were prepared using: (1) 1 mL of human milk from one donor (adulterated at 10%)+25 mL of PBS and (2) 1 mL of milk from ten donors combined (100 μL each, with one of them adulterated at 10%)+1.6 mL PBS. The final concentration of adulterant in PBS is the same in both samples.

Adulteration levels compared were 0% and 5% for all adulterants, as well as 10% Goat Milk and 10% Soy Formula as the cutoff calibrators in the Veratox® Total Milk Allergen and Veratox® Soy Allergen kits respectively. A 20% Goat Milk adulteration sample was also included in the Veratox® Total Milk Allergen kit. The samples were prepared using the assay volumes described above, and the extraction step was omitted. The subsequent dilution for both ELISA assays was 1:100 for all samples.

The highest standard in each kit was included in the assay as a positive control, and PBS and 100% human breast milk were included as negative controls. The cutoff calibrators were analyzed in triplicate and all samples in duplicate. Sample analyses were repeated if the % CV of the replicates exceeded 15%.

Samples were analyzed with the appropriate ELISA assay. Samples were washed using an automatic plate washer (ten times for the Veratox® Total Milk Allergen kit and five times for the Veratox® Soy Allergen kit). Optical densities were measured using an Epoch plate reader at 650 nm.

The results from the Veratox® Total Milk Allergen kit are summarized in Table 3.

TABLE 3
Veratox ® Total Milk ELISA Assay

| | | 1 Donor/ | 10 Donors/ | |
|---|---|---|---|---|
| | | Sample: | Sample: | |
| | | 1 mL | 100 μL each | |
| | | 1 mL + 25 mL | 1 mL + 1.6 mL | Percent |
| Run | Sample | (PBS) | (PBS) | Expected |
| 1 | 5% Goat Milk | 0.489 | 0.329 | 67.3 |
| 2 | 5% Goat Milk | 0.364 | 0.205 | 56.3 |
| 1 | 10% Goat Milk | 0.660 | 0.389 | 58.9 |
| 2 | 10% Goat Milk | 0.669 | 0.345 | 51.6 |
| 2 | 20% Goat Milk | 0.845 | 0.488 | 57.8 |
| | | | AVG. | 58.4 |
| | | | STD. DEV. | 5.7 |
| | | | % CV | 9.8 |
| 1 | 5% Cow Milk | 1.605 | 1.494 | 93.1 |
| 2 | 5% Cow Milk | 1.182 | 1.009 | 85.4 |
| 2 | 10% Cow Milk | 1.971 | 1.808 | 91.7 |
| | | | AVG. | 90.1 |
| | | | STD. DEV. | 4.1 |
| | | | % CV | 4.6 |
| 2 | 5% Cow Formula | 0.785 | 0.679 | 86.5 |
| 2 | 5% Cow Formula | 0.791 | 0.692 | 87.5 |
| 2 | 10% Cow Formula | 1.543 | 1.430 | 92.7 |
| 2 | 10% Cow Formula | 1.453 | 1.433 | 98.6 |
| | | | AVG. | 91.3 |
| | | | STD. DEV. | 5.6 |
| | | | % CV | 6.1 |
| 1 | NEG. OD | 0.157 | 0.172 | 109.6 |

When pooling ten donors per sample and decreasing sample volume to 100 μL per donor, the absorbance values (OD) obtained with Cow Milk and Cow Formula were similar to those by the original assay conditions (˜10% reduction in OD).

In contrast, the Goat Milk results were different from the original assay conditions and the reduction in absorbance values (OD) was ˜40%. There may be a competitive binding of the antibody on the ELISA plate between antigens in goat milk and antigens in human breast milk. When ten donors were pooled, the ratio of the human breast milk to the adulterant changed (Table 4). The data suggest that the extent of binding of the antibody on the ELISA plate to the antigens in goat milk is reduced in the presence of an increased amount of breast milk, culminating in a reduced OD. Where the 10% Goat Milk cutoff calibrator previously was ten standard deviations above the negative control, at the reduced values, the 10% Goat Milk cutoff calibrator was approximately five standard deviations above the negative control.

TABLE 4
Comparison of Donor and Adulterant Volumes Used

| | | | | | Adulteration of 1 | | Total in final 1 | | Ratio |
|---|---|---|---|---|---|---|---|---|---|
| | # of | Volume | | Total | donor | | mL | | Breast |
| Assay | donors | per | | Sample | Breast | Goat | Breast | Goat | to Goat |
| Volumes | per well | donor | | Volume | Milk | Milk | Milk | Milk | Milk |
| 1 | 1 | 1 | mL | 1 mL | 900 μL | 100 μL | 900 μL | 100 μL | 9:1 |
| 2 | 10 | 100 | μL | 1 mL | 90 μL | 10 μL | 990 μL | 10 μL | 99:1 |

In order to determine the adulteration level at which Cow Milk and Cow Formula tested negative, serial dilutions (1%, 0.5%, 0.25%, 0.125%, and 0.063%) of each adulterant in a ten-donor breast milk pool (10 donors/sample at 100 μL each) were analyzed in singlet, and compared to the 10% Goat Milk cutoff calibrator (Table 5).

TABLE 5
Absorbance Values of Various Adulteration Levels of Cow Milk and
Cow Formula in the Veratox ® Total Milk ELISA Assay

| | Level of Adulteration | | | | |
|---|---|---|---|---|---|
| | 1% | 0.5% | 0.25% | 0.125% | 0.063% |
| Adulterant | | | | | |
| Cow Milk | 0.473 | 0.298 | 0.195 | 0.144 | 0.124 |
| Cow Formula | 0.363 | 0.232 | 0.158 | 0.130 | 0.122 |

| | |
|---|---|
| Cutoff Calibrator | 0.285 |
| (10% Goat Milk) | |
| NEG. OD | 0.103 |

The Veratox® Total Milk Allergen kit was found to be highly responsive to Cow Milk and Cow Formula adulteration. Levels of 0.5% Cow Milk and 1% Cow Formula generated greater OD values than when the 10% Goat Milk cutoff calibrator was used.

The results of the Veratox® Soy Allergen kit are summarized in Table 6.

TABLE 6
Veratox ® Soy ELISA Assay

| | | 1 Donor/sample: | 10 Donors/sample: | |
|---|---|---|---|---|
| | | 1 mL | 100 μL each | |
| | | 1 mL + 25 mL | 1 mL + 1.6 mL | Percent |
| Run | Sample | (PBS) | (PBS) | Expected |
| 1 | 5% Soy Formula | 0.198 | 0.220 | 111.1 |
| 1 | 10% Soy Formula | 0.364 | 0.389 | 106.9 |
| 1 | 5% Soy Milk | 0.319 | 0.316 | 99.1 |
| 1 | NEG. OD | 0.067 | 0.073 | 109.0 |
| | | | AVG. | 106.5 |
| | | | STD. DEV. | 5.3 |
| | | | % CV | 4.9 |

When pooling ten donors per sample and decreasing sample volume to 100 μL per donor, the absorbance values (OD) of Soy Milk and Soy Formula were similar to those obtained under the original assay conditions.

In summary, the results demonstrated that for Cow Milk, Cow Formula, Soy Milk and Soy Formula, pooling donors (ten donors/sample) and further decreasing donor volumes (100 μL each donor) generated data equivalent to the assay conditions of 1 mL of sample per donor and one donor per test sample were used. As described above, the absorbance value for Goat Milk was reduced by approximately 40%. The Veratox® Total Milk Allergen kit was highly responsive to both Cow Milk and Cow Formula and can detect adulteration levels of 1% as positive.

### Precision and Accuracy Testing

The precision and accuracy of the method using 10 donors per sample at 100 μL each was further evaluated. The precision of the method was analyzed twice for each of the cutoff calibrators and all ten donors were analyzed individually, to determine 1) intra-donor and inter-donor variations, and 2) assay precision. Individual samples of human breast milk from ten donors were spiked with 10% Goat Milk or 5% Soy Formula. Each donor was analyzed in duplicate, and absorbance values (OD) were obtained ten times over an approximate 12 minute time period.

The average absorbance value (OD), standard deviation (SD), and % CV were calculated for each adulterant, donor, and run. In all cases for the Veratox® Total Milk Allergen and Veratox® Soy Allergen kits, the intra-donor variation was very small, and the inter-donor variation and the assay precision were less than 10%. The data are summarized in Tables 7 and 8 respectively.

TABLE 7
Precision Validation Data for the Veratox ® Total Milk ELISA Assay

| Time | Donors | | | | | |
|---|---|---|---|---|---|---|
| (min) | 15607 | 15966 | 16226 | 16528 | 16580 | 17046 |

RUN 1

| | | | | | | | | | | | | |
|---|---|---|---|---|---|---|---|---|---|---|---|---|
| 8 | 0.328 | 0.323 | 0.310 | 0.320 | 0.336 | 0.323 | 0.306 | 0.288 | 0.359 | 0.351 | 0.278 | 0.291 |
| 9 | 0.328 | 0.321 | 0.310 | 0.321 | 0.337 | 0.325 | 0.305 | 0.288 | 0.356 | 0.351 | 0.277 | 0.291 |
| 11 | 0.328 | 0.321 | 0.310 | 0.321 | 0.338 | 0.326 | 0.305 | 0.290 | 0.355 | 0.352 | 0.277 | 0.291 |
| 12.67 | 0.329 | 0.322 | 0.312 | 0.322 | 0.340 | 0.327 | 0.306 | 0.292 | 0.355 | 0.354 | 0.278 | 0.292 |
| 13.5 | 0.331 | 0.323 | 0.313 | 0.323 | 0.341 | 0.328 | 0.307 | 0.294 | 0.357 | 0.356 | 0.280 | 0.294 |
| 14.75 | 0.333 | 0.325 | 0.314 | 0.324 | 0.342 | 0.329 | 0.307 | 0.295 | 0.356 | 0.356 | 0.282 | 0.296 |
| 16 | 0.334 | 0.326 | 0.315 | 0.325 | 0.342 | 0.330 | 0.308 | 0.296 | 0.355 | 0.357 | 0.283 | 0.298 |
| 17.5 | 0.335 | 0.327 | 0.315 | 0.325 | 0.342 | 0.330 | 0.309 | 0.297 | 0.353 | 0.356 | 0.284 | 0.298 |
| 18.75 | 0.335 | 0.327 | 0.315 | 0.326 | 0.342 | 0.330 | 0.308 | 0.297 | 0.352 | 0.356 | 0.284 | 0.299 |
| 20 | 0.334 | 0.327 | 0.315 | 0.327 | 0.342 | 0.330 | 0.308 | 0.297 | 0.351 | 0.354 | 0.285 | 0.299 |
| AVG | 0.332 | 0.324 | 0.313 | 0.323 | 0.340 | 0.328 | 0.307 | 0.293 | 0.355 | 0.354 | 0.281 | 0.295 |
| SD | 0.003 | 0.002 | 0.002 | 0.002 | 0.002 | 0.002 | 0.001 | 0.004 | 0.002 | 0.002 | 0.003 | 0.003 |
| % CV | 0.9 | 0.8 | 0.7 | 0.7 | 0.7 | 0.8 | 0.4 | 1.2 | 0.7 | 0.6 | 1.1 | 1.2 |

RUN 2

| 7 | 0.320 | 0.323 | 0.309 | 0.436 | 0.327 | 0.338 | 0.321 | 0.305 | 0.323 | 0.356 | 0.322 | 0.326 |
|---|---|---|---|---|---|---|---|---|---|---|---|---|
| 8.5 | 0.320 | 0.322 | 0.311 | 0.436 | 0.326 | 0.338 | 0.320 | 0.307 | 0326 | 0.357 | 0.321 | 0.324 |
| 9.75 | 0.321 | 0.324 | 0.315 | 0.439 | 0.327 | 0.339 | 0.320 | 0.307 | 0.330 | 0.358 | 0.321 | 0.323 |
| 11.25 | 0.323 | 0.328 | 0.319 | 0.442 | 0.329 | 0.340 | 0.321 | 0.308 | 0.334 | 0.360 | 0.322 | 0.323 |
| 12.5 | 0.327 | 0.332 | 0.322 | 0.446 | 0.330 | 0.341 | 0.322 | 0.310 | 0.338 | 0.361 | 0.324 | 0.324 |
| 13.75 | 0.331 | 0.334 | 0.324 | 0.449 | 0.331 | 0.342 | 0.323 | 0.311 | 0.342 | 0.363 | 0.325 | 0.325 |
| 15 | 0.333 | 0.335 | 0.326 | 0.451 | 0.332 | 0.343 | 0.322 | 0.311 | 0.344 | 0.363 | 0.325 | 0.324 |
| 16.25 | 0.334 | 0.336 | 0.327 | 0.452 | 0.332 | 0.343 | 0.322 | 0.311 | 0.346 | 0.363 | 0.325 | 0.324 |
| 17.75 | 0.334 | 0.335 | 0.328 | 0.453 | 0.333 | 0.343 | 0.321 | 0.311 | 0.346 | 0.363 | 0.325 | 0.323 |
| 19 | 0.335 | 0.335 | 0.329 | 0.453 | 0.333 | 0.344 | 0.321 | 0.311 | 0.347 | 0.363 | 0.325 | 0.323 |
| AVG | 0.328 | 0.331 | 0.322 | 0.446 | 0.330 | 0.341 | 0.321 | 0.309 | 0.338 | 0.361 | 0.324 | 0.324 |
| SD | 0.006 | 0.005 | 0.007 | 0.007 | 0.003 | 0.002 | 0.001 | 0.002 | 0.009 | 0.003 | 0.002 | 0.001 |
| % CV | 1.9 | 1.7 | 2.3 | 1.5 | 0.8 | 0.7 | 0.4 | 0.7 | 2.6 | 0.7 | 0.5 | 0.3 |
| | Time | Donors | | | | | | | | | | |
| | (min) | 17076 | | 17193 | | 17363 | | 17617 | | AVG | SD | % CV |
| | RUN 1 | | | | | | | | | | | |
| | 8 | 0.292 | 0.290 | 0.272 | 0.276 | 0.292 | 0.267 | 0.302 | 0.295 | 0.305 | 0.026 | 8.5 |
| | 9 | 0.292 | 0.292 | 0.273 | 0.276 | 0.294 | 0.269 | 0.303 | 0.297 | 0.305 | 0.025 | 8.2 |
| | 11 | 0.293 | 0.294 | 0.274 | 0.278 | 0.296 | 0.271 | 0.305 | 0.300 | 0.306 | 0.025 | 8.1 |
| | 12.67 | 0.294 | 0.297 | 0.276 | 0.280 | 0.298 | 0.272 | 0.307 | 0.303 | 0.308 | 0.025 | 8.0 |
| | 13.5 | 0.296 | 0.298 | 0.277 | 0281 | 0.300 | 0.273 | 0.309 | 0.305 | 0.309 | 0.025 | 8.0 |
| | 14.75 | 0.299 | 0.299 | 0.278 | 0.282 | 0.301 | 0.274 | 0.310 | 0.308 | 0.310 | 0.024 | 7.8 |
| | 16 | 0.299 | 0.300 | 0.279 | 0.283 | 0.302 | 0.275 | 0.311 | 0.310 | 0.311 | 0.024 | 7.7 |
| | 17.5 | 0.299 | 0.301 | 0.279 | 0.283 | 0.302 | 0.275 | 0.312 | 0.311 | 0.312 | 0.024 | 7.6 |
| | 18.75 | 0.299 | 0301 | 0.279 | 0.283 | 0.303 | 0.275 | 0.312 | 0.313 | 0.312 | 0.024 | 7.6 |
| | 20 | 0.299 | 0.301 | 0.279 | 0.284 | 0.303 | 0.275 | 0.312 | 0.313 | 0.312 | 0.023 | 7.4 |
| | AVG | 0.296 | 0.297 | 0.277 | 0.281 | 0.299 | 0.273 | 0.308 | 0.306 | Over- | AVG | 0.309 |
| | SD | 0.003 | 0.004 | 0.003 | 0.003 | 0.004 | 0.003 | 0.004 | 0.007 | all | SD | 0.024 |
| | % CV | 1.0 | 1.3 | 1.0 | 1.1 | 1.3 | 1.0 | 1.2 | 2.1 | | % CV | 7.8 |
| | RUN 2 | | | | | | | | | | | |
| | 7 | 0.285 | 0.277 | 0.300 | 0.305 | 0.320 | 0.307 | 0.323 | 0.324 | 0.322 | 0.032 | 9.9 |
| | 8.5 | 0.287 | 0.275 | 0.301 | 0.306 | 0.321 | 0.307 | 0.324 | 0.325 | 0.323 | 0.032 | 9.8 |
| | 9.75 | 0.288 | 0.277 | 0.303 | 0309 | 0.322 | 0.307 | 0.323 | 0.325 | 0.324 | 0.032 | 9.9 |
| | 11.25 | 0.289 | 0.279 | 0.305 | 0.311 | 0.324 | 0.309 | 0.324 | 0.327 | 0.326 | 0.032 | 9.9 |
| | 12.5 | 0.293 | 0.281 | 0.308 | 0.314 | 0.325 | 0.311 | 0.326 | 0.329 | 0.328 | 0.032 | 9.9 |
| | 13.75 | 0.297 | 0.284 | 0.310 | 0.316 | 0.327 | 0.313 | 0.329 | 0.331 | 0.330 | 0.033 | 9.9 |
| | 15 | 0.299 | 0.286 | 0.311 | 0.318 | 0.328 | 0.315 | 0.331 | 0.333 | 0.332 | 0.033 | 9.8 |
| | 16.25 | 0.300 | 0.287 | 0.312 | 0.319 | 0.329 | 0.316 | 0.331 | 0.333 | 0.332 | 0.033 | 9.8 |
| | 17.75 | 0.301 | 0.288 | 0.313 | 0.319 | 0.330 | 0.318 | 0.332 | 0.333 | 0.332 | 0.033 | 9.8 |
| | 19 | 0.301 | 0.288 | 0.313 | 0.320 | 0.331 | 0.320 | 0.332 | 0.333 | 0.333 | 0.033 | 9.8 |
| | AVG | 0.295 | 0.283 | 0.308 | 0.314 | 0.326 | 0.313 | 0.328 | 0.330 | Over- | AVG | 0.329 |
| | SD | 0.006 | 0.005 | 0.005 | 0.006 | 0.004 | 0.005 | 0.004 | 0.004 | all | SD | 0.032 |
| | % CV | 2.2 | 1.7 | 1.6 | 1.8 | 1.3 | 1.7 | 1.2 | 1.2 | | % CV | 9.7 |

TABLE 8
Precision Validation Data for the Veratox ® Soy ELISA Assay

| Time | Donors | | | | | |
|---|---|---|---|---|---|---|
| (min) | 15607 | 15966 | 16226 | 16528 | 16580 | 17046 |

RUN 1

| | | | | | | | | | | | | |
|---|---|---|---|---|---|---|---|---|---|---|---|---|
| 7.5 | 0.311 | 0.301 | 0.299 | 0.306 | 0.213 | 0.270 | 0.275 | 0.279 | 0.253 | 0.245 | 0.250 | 0.257 |
| 9 | 0.311 | 0.302 | 0.299 | 0.306 | 0.280 | 0.270 | 0.274 | 0.279 | 0.253 | 0.245 | 0.249 | 0.257 |
| 10.25 | 0.310 | 0.302 | 0.299 | 0.305 | 0.280 | 0.271 | 0.274 | 0.278 | 0.253 | 0.245 | 0.249 | 0.258 |
| 11.5 | 0.310 | 0.302 | 0.299 | 0.304 | 0.279 | 0.271 | 0.274 | 0.278 | 0.253 | 0.245 | 0.249 | 0.258 |
| 12.75 | 0.310 | 0.302 | 0.298 | 0.304 | 0.279 | 0.271 | 0.273 | 0.278 | 0.253 | 0.245 | 0.249 | 0.259 |
| 14 | 0.310 | 0.302 | 0.293 | 0.303 | 0.278 | 0.271 | 0.274 | 0.277 | 0.253 | 0.246 | 0.242 | 0.259 |
| 15.25 | 0.310 | 0.302 | 0.297 | 0.303 | 0.278 | 0.271 | 0.273 | 0.277 | 0.252 | 0.246 | 0.248 | 0.260 |
| 16.5 | 0.310 | 0.302 | 0.297 | 0.303 | 0.278 | 0.271 | 0.273 | 0.277 | 0.252 | 0.246 | 0.248 | 0.260 |
| 18 | 0.310 | 0.302 | 0.296 | 0.302 | 0.277 | 0.271 | 0.273 | 0.277 | 0.252 | 0.246 | 0.248 | 0.261 |
| 19.12 | 0.309 | 0.302 | 0.296 | 0.302 | 0.277 | 0.271 | 0.272 | 0.277 | 0.252 | 0.247 | 0.247 | 0.261 |
| AVG | 0.310 | 0.302 | 0.298 | 0.304 | 0.279 | 0.271 | 0.274 | 0278 | 0.253 | 0.246 | 0.249 | 0.259 |
| SD | 0.001 | 0.000 | 0.001 | 0.001 | 0.001 | 0.000 | 0.001 | 0.001 | 0.001 | 0.001 | 0.001 | 0.001 |
| % CV | 0.2 | 0.1 | 0.4 | 0.5 | 0.5 | 0.2 | 0.3 | 0.3 | 0.2 | 0.3 | 0.3 | 0.6 |

RUN 2

| 7.25 | 0.213 | 0.250 | 0.216 | 0.218 | 0.238 | 0.226 | 0.234 | 0.247 | 0.241 | 0.246 | 0.249 | 0.255 |
|---|---|---|---|---|---|---|---|---|---|---|---|---|
| 8.75 | 0.214 | 0.250 | 0.217 | 0.219 | 0.238 | 0.226 | 0.233 | 0.245 | 0.240 | 0.245 | 0.249 | 0.255 |
| 10 | 0.213 | 0.250 | 0.218 | 0.220 | 0.238 | 0.225 | 0.231 | 0.244 | 0.239 | 0.244 | 0.248 | 0.254 |
| 11.3 | 0.216 | 0.252 | 0.218 | 0.221 | 0.237 | 0.224 | 0.230 | 0.245 | 0.240 | 0.243 | 0.247 | 0.255 |
| 12.67 | 0.218 | 0.253 | 0.220 | 0.222 | 0.236 | 0.224 | 0.230 | 0.247 | 0.243 | 0.245 | 0.249 | 0.258 |
| 13.9 | 0.212 | 0.254 | 0.226 | 0.227 | 0.238 | 0.230 | 0.235 | 0.252 | 0.249 | 0.250 | 0.252 | 0.261 |
| 15 | 0.213 | 0.254 | 0.220 | 0.222 | 0.236 | 0.224 | 0.230 | 0.247 | 0.243 | 0.245 | 0.249 | 0.258 |
| 16.3 | 0.218 | 0.254 | 0.230 | 0.232 | 0.241 | 0.233 | 0.237 | 0.254 | 0.251 | 0.251 | 0.253 | 0.261 |
| 17.5 | 0.218 | 0.254 | 0.233 | 0.235 | 0.242 | 0.235 | 0.238 | 0.255 | 0.252 | 0.253 | 0.253 | 0.262 |
| 18.75 | 0.218 | 0.255 | 0.236 | 0.239 | 0.244 | 0.236 | 0.239 | 0.255 | 0.752 | 0.253 | 0.253 | 0.262 |
| AVG | 0.216 | 0.253 | 0.223 | 0.226 | 0.239 | 0.228 | 0.234 | 0.249 | 0.245 | 0.248 | 0.250 | 0.258 |
| SD | 0.002 | 0.002 | 0.007 | 0.007 | 0.003 | 0.005 | 0.003 | 0.004 | 0.005 | 0.004 | 0.002 | 0.003 |
| % CV | 1.1 | 0.8 | 3.3 | 3.3 | 1.1 | 2.1 | 1.5 | 1.8 | 2.2 | 1.6 | 0.9 | 1.2 |
| | Time | Donors | | | | | | | | | | |
| | (min) | 17076 | | 17193 | | 17363 | | 17617 | | AVG | SD | % CV |
| | RUN 1 | | | | | | | | | | | |
| | 7.5 | 0.301 | 0.270 | 0.290 | 0.288 | 0.282 | 0.267 | 0.262 | 0.255 | 0.277 | 0.020 | 7.2 |
| | 9 | 0.301 | 0.269 | 0.290 | 0.289 | 0.281 | 0.267 | 0.262 | 0.255 | 0.277 | 0.020 | 7.3 |
| | 10.25 | 0.301 | 0.269 | 0.290 | 0.289 | 0.281 | 0.267 | 0.263 | 0.255 | 0.277 | 0.020 | 7.2 |
| | 11.5 | 0.300 | 0.269 | 0.289 | 0.289 | 0.281 | 0.267 | 0.263 | 0.256 | 0.277 | 0.020 | 7.1 |
| | 12.75 | 0.300 | 0.269 | 0.288 | 0.269 | 0.281 | 0.267 | 0.264 | 0.257 | 0.277 | 0.019 | 7.0 |
| | 14 | 0.300 | 0.266 | 0.288 | 0.229 | 0.281 | 0.267 | 0.264 | 0.257 | 0.277 | 0.019 | 7.0 |
| | 15.25 | 0.300 | 0.268 | 0.288 | 0.289 | 0.280 | 0.267 | 0.264 | 0.258 | 0.277 | 0.019 | 7.0 |
| | 16.5 | 0.300 | 0.268 | 0.287 | 0.289 | 0.280 | 0.266 | 0.265 | 0.258 | 0.277 | 0.019 | 7.0 |
| | 18 | 0.300 | 0.268 | 0.287 | 0.289 | 0.280 | 0.266 | 0.265 | 0.258 | 0.276 | 0.019 | 6.9 |
| | 19.12 | 0.301 | 0.268 | 0.287 | 0.289 | 0.280 | 0.266 | 0.265 | 0.258 | 0.276 | 0.019 | 6.9 |
| | AVG | 0.300 | 0.269 | 0.288 | 0.289 | 0.281 | 0.267 | 0.264 | 0.257 | Overall | AVG | 0.277 |
| | SD | 0.001 | 0.001 | 0.001 | 0.000 | 0.001 | 0.000 | 0.001 | 0.001 | | SD | 0.019 |
| | % CV | 0.2 | 0.3 | 0.4 | 0.1 | 0.2 | 0.2 | 0.4 | 0.5 | | % CV | 769 |
| | RUN 2 | | | | | | | | | | | |
| | 7.25 | 0.231 | 0.220 | 0.200 | 0.215 | 0.225 | 0.203 | 0.208 | 0.206 | 0.227 | 0.017 | 7.6 |
| | 8.75 | 0.231 | 0.220 | 0.201 | 0.215 | 0.224 | 0.204 | 0.208 | 0.207 | 0.227 | 0.017 | 7.3 |
| | 10 | 0.232 | 0.221 | 0.202 | 0.215 | 0.222 | 0.204 | 0.208 | 0.207 | 0.227 | 0.016 | 7.2 |
| | 11.3 | 0.233 | 0.224 | 0.204 | 0.216 | 0.223 | 0.205 | 0.208 | 0.207 | 0.227 | 0.016 | 7.1 |
| | 12.67 | 0.235 | 0.227 | 0.207 | 0.219 | 0.227 | 0.208 | 0.211 | 0.212 | 0.230 | 0.016 | 6.8 |
| | 13.9 | 0.237 | 0.232 | 0.212 | 0.225 | 0.235 | 0.216 | 0.219 | 0.220 | 0.234 | 0.014 | 6.2 |
| | 15 | 0.235 | 0.227 | 0.207 | 0.219 | 0.227 | 0.208 | 0.211 | 0.212 | 0.229 | 0.016 | 6.9 |
| | 16.3 | 0.238 | 0.233 | 0.214 | 0.226 | 0.236 | 0.218 | 0.222 | 0.222 | 0.236 | 0.014 | 5.9 |
| | 17.5 | 0.238 | 0.234 | 0.214 | 0.227 | 0.237 | 0.219 | 0.224 | 0.223 | 0.237 | 0.014 | 5.9 |
| | 18.75 | 0.238 | 0.235 | 0.215 | 0.227 | 0.237 | 0.219 | 0.225 | 0.224 | 0.238 | 0.014 | 5.8 |
| | AVG | 0.235 | 0.227 | 0.208 | 0.220 | 0.229 | 0.210 | 0.214 | 0.214 | Overall | AVG | 0.231 |
| | SD | 0.003 | 0.006 | 0.006 | 0.005 | 0.006 | 0.007 | 0.007 | 0.007 | | SD | 0.016 |
| | % CV | 1.2 | 2.6 | 2.8 | 2.4 | 2.7 | 3.2 | 3.4 | 3.5 | | % CV | 6.8 |

The accuracy, or sensitivity and specificity, of an analytical method are the closeness of test results obtained by that method to the true result. The ability of each assay to correctly determine the true positives and negatives was examined.

The sensitivity of a test refers to the ability of that test to correctly identify true positives and is calculated using the following equation: Sensitivity=(True positives)/(True positives+False negatives). The specificity of a test refers to the ability of the test to correctly identify true negatives and is calculated using the following equation:

Specificity=(True negatives)/(True negatives+False positives).

The accuracy of the method was analyzed twice for each adulterant. Pools of ten donors were prepared (100 μL each) in which one of the donor samples was adulterated at the level indicated. The adulterated donor in a pool was rotated. Samples were spiked with the adulterants at the following levels for the Veratox® Total Milk Allergen kit: Goat Milk (5%, 10% (cutoff calibrator), and 20%), Cow Milk (0.25% and 10%), Cow Formula (0.25% and 10%), and Negative Control (0%).

To make a positive or negative determination, the average absorbance value of each sample (duplicate) was compared to the average absorbance value obtained for the respective cutoff calibrator (triplicate) of the assay. If the sample absorbance value is less than the cutoff absorbance, the result is negative. If the sample absorbance value is greater than the cutoff absorbance, the result is positive.

Using the Veratox® Total Milk Allergen kit, adulterated and unadulterated breast milk samples were analyzed. 10% Goat Milk (bold) was used as the cutoff calibrator. The average absorbance values are presented in Table 9. When the data were rejected due to replicate sample % CV exceeding 15%, the sample analyses were repeated and the average absorbance values (italic) were determined

TABLE 9A
Accuracy of Adulteration with Goat Milk for the Veratox ®
Total Milk ELISA Assay

| | | Run 1 | | | | Run 2 | | | |
|---|---|---|---|---|---|---|---|---|---|
| Donors | | Adulteration | | | | Adulteration | | | |
| ID | Number | 0% | 5% | 10% | 20% | 0% | 5% | 10% | 20% |
| A.1 | 15607 | 0.130 | 0.247 | 0.325 | 0.424 | 0.126 | 0.243 | 0.323 | 0.507 |
| A.2 | 15966 | 0.136 | 0.215 | 0.315 | 0.430 | 0.126 | 0.213 | [symbol] | 0.446 |
| B | 16226 | 0.141 | 0.219 | 0.331 | 0.477 | 0.157 | 0.199 | 0.333 | 0.432 |
| C | 16528 | 0.131 | 0.209 | 0.297 | 0.480 | 0.141 | 0.202 | 0.314 | 0.431 |
| D | 16580 | 0.142 | 0.216 | 0.354 | 0.430 | 0.134 | 0.196 | 0.344 | 0.458 |
| E | 17046 | 0.155 | 0.204 | 0.284 | 0.384 | 0.136 | 0.204 | 0.322 | 0.437 |
| F | 17076 | 0.134 | 0.215 | 0.292 | 0.408 | 0.132 | 0.228 | 0.306 | 0.414 |
| G | 17193 | 0.155 | 0.204 | 0.274 | 0.401 | 0.122 | 0.225 | 0.315 | 0.401 |
| H | 17363 | 0.146 | 0.189 | 0.281 | 0.447 | 0.162 | 0.225 | 0.324 | 0.409 |
| I | 17617 | 0.141 | 0.193 | 0.300 | 0.404 | 0.139 | 0.209 | 0.310 | 0.396 |
| | AVG. | 0.141 | 0.211 | 0.305 | 0.429 | 0.138 | 0.214 | 0.320 | 0.433 |
| | STD. | 0.009 | 0.016 | 0.025 | 0.032 | 0.013 | 0.015 | 0.012 | 0.033 |
| | DEV. | | | | | | | | |
| | % CV | 6.3 | 7.6 | 8.3 | 7.4 | 9.5 | 7.1 | 3.8 | 7.5 |

| | | |
|---|---|---|
| Pool C/O Calibrator*: | 0.309 | 0.327 |

| | | |
|---|---|---|
| Number of Correct Results | 30/30 | 30/30 |
| Accuracy | 100% | 100% |

*A cutoff calibrator created from a pool of ten donors contributing equal volumes.

TABLE 9B
Accuracy of Adulteration with Cow Milk for the Veratox ® Total Milk ELISA Assay

| | | Run 1 | | | Run 2 | | |
|---|---|---|---|---|---|---|---|
| | | Adulteration | | | Adulteration | | |
| Donors | | | C/O | | | C/O | |
| ID | Number | 0.25% | Cal* | 10% | 0.25% | Cal* | 10% |
| A.1 | 15607 | 0.221 | 0.317 | 1.388 | 0.237 | 0.313 | 1.508 |
| A.2 | 15966 | 0.234 | | 1.378 | 0.246 | | 1.391 |
| B | 16226 | 0.214 | | 1.295 | 0.238 | | 1.458 |
| C | 16528 | 0.219 | | 1.425 | 0.241 | | 1.346 |
| D | 16580 | 0.224 | | 1.295 | 0.248 | | 1.323 |
| E | 17046 | 0.221 | | 1.300 | 0.234 | | 1.312 |
| F | 17076 | 0.223 | | 1.130 | 0.258 | | 1.446 |
| G | 17193 | 0.229 | | 1.196 | 0.243 | | 1.342 |
| H | 17363 | 0.222 | | 1.125 | 0.248 | | 1.324 |
| I | 17617 | 0.243 | | 1.353 | 0.232 | | 1.282 |
| | AVG. | 0.225 | | 1.289 | 0.243 | | 1.373 |
| | STD. | 0.008 | | 0.106 | 0.008 | | 0.074 |
| | DEV. | | | | | | |
| | % CV | 3.7 | | 8.2 | 3.2 | | 5.4 |
| | NEG. | | 0.134 | | | 0.155 | |
| | OD: | | | | | | |

| | | |
|---|---|---|
| NUMBER of CORRECT | 20/20 | 20/20 |
| RESULTS | | |
| ACCURACY | 100% | 100% |

*Cutoff calibrator (10% Goat Milk in a pool of ten donors, with one donor adulterated)

TABLE 9C
Accuracy of Adulteration with Cow Formula for the Veratox ® Total Milk ELISA
Assay

| | | Run 1 | | | Run 2 | | |
|---|---|---|---|---|---|---|---|
| Donors | | Adulteration | | | Adulteration | | |
| ID | Number | 0.25% | C/O Cal* | 10% | 0.25% | C/O Cal* | 10% |
| A.1 | 15607 | 0.188 | 0.317 | 1.178 | 0.202 | 0.313 | 1.261 |
| A.2 | 15966 | 0.179 | | 1.086 | 0.188 | | 1.157 |
| B | 16226 | 0.180 | | 0.987 | 0.186 | | 1.242 |
| C | 16528 | 0.167 | | 1.057 | 0.193 | | 1.197 |
| D | 16580 | 0.163 | | 1.005 | 0.195 | | 1.221 |
| E | 17046 | 0.182 | | 1.308 | 0.193 | | 1.237 |
| F | 17076 | 0.184 | | 1.064 | 0.203 | | 1.195 |
| G | 17193 | 0.185 | | 1.021 | 0.190 | | 1.202 |
| H | 17363 | 0.169 | | 1.032 | 0.198 | | 1.270 |
| I | 17617 | 0.167 | | 1.025 | 0.186 | | 1.258 |
| | AVG. | 0.176 | | 1.076 | 0.193 | | 1.224 |
| | STD. DEV. | 0.009 | | 0.097 | 0.006 | | 0.036 |
| | % CV | 5.1 | | 9.0 | 3.2 | | 2.9 |
| | NEG. OD: | | 0.134 | | | 0.155 | |

| | | |
|---|---|---|
| NUMBER of CORRECT RESULTS | 20/20 | 20/20 |
| ACCURACY | 100% | 100% |

*Cutoff calibrator (10% Goat Milk in a pool of ten donors, with one donor adulterated)

In summary, the accuracy of the Veratox® Total Milk ELISA assay in detecting adulteration with Goat Milk, Cow Milk, and Cow Formula was 100%.

Using the Veratox® Soy Allergen kit, adulterated and unadulterated breast milk samples were analyzed using 10% Soy Formula (bold) as the cutoff calibrator. The average absorbance values are presented in Table 10. The data were rejected if the replicate sample % CV exceeded 15%.

TABLE 10
Accuracy of Adulteration with Soy Formula for the Veratox ®
Soy ELISA Assay

| Donor | | % Adulteration | | | |
|---|---|---|---|---|---|
| ID | Number | 0% | 5% | 10% | 15% |
| A.1 | 15607 | 0.059 | 0.212 | 0.405 | 0.451 |
| A.2 | 15966 | 0.060 | 0.207 | 0.390 | 0.547 |
| B | 16226 | * | 0.217 | 0.419 | 0.525 |
| C | 16528 | 0.061 | 0.220 | 0.415 | 0.565 |
| D | 16580 | 0.062 | 0.216 | 0.381 | 0.597 |
| E | 17046 | 0.064 | 0.229 | 0.393 | 0.646 |
| F | 17076 | 0.062 | 0.207 | 0.407 | 0.579 |
| G | 17193 | 0.061 | 0.220 | 0.390 | 0.533 |
| H | 17363 | 0.063 | 0.211 | 0.387 | 0.549 |
| I | 17617 | 0.076 | 0.219 | 0.390 | 0.559 |
| | AVG. | 0.063 | 0.216 | 0.398 | 0.555 |
| | STD | 0.005 | 0.007 | 0.013 | 0.051 |
| | DEV. | | | | |
| | % CV | 8.0 | 3.1 | 3.2 | 9.1 |

| | |
|---|---|
| NUMBER OF CORRECT | 29/29 |
| RESULTS | |
| ACCURACY | 100% |

*Data point excluded because sample duplicates exceeded a % CV of 15%

In Table 10, the specificity (correct identification of true negatives) was 100% for both cases. Sensitivity (correct identification of true positives) was 100% when using the average of the duplicate wells analyzed per donor. When considering individual well data as shown in Table 11, sensitivity was 95%. In Table 11, the OD reading of the replicate in well 1 of donor A.1 (adulterated at 15%, italics), is lower than the three highest values (underlined) obtained with the 10% cutoff calibrator (Donor B, well 1; Donor C, well 1; Donor F, well 1), and this generated a false negative. This data point was also very close (≦0.004 OD) to four data points in the cutoff calibrator group. As a result, the adulteration level of the cutoff calibrator for the Soy Allergen assay was decreased from 10% Soy Formula to 5% Soy Formula.

TABLE 11
Accuracy Validation Data of Adulteration with Soy Formula for the
Veratox ® Soy ELISA Assay

| Donor | | | % Adulteration | | | |
|---|---|---|---|---|---|---|
| ID | Number | Well | 0% | 5% | 10% | 15% |
| A.1 | 15607 | 1 | 0.057 | 0.220 | 0.414 | 0.418 |
| | | 2 | 0.061 | 0.204 | 0.396 | 0.484 |
| A.2 | 15966 | 1 | 0.061 | 0.213 | 0.415 | 0.567 |
| | | 2 | 0.060 | 0.201 | 0.365 | 0.527 |
| B | 16226 | 1 | 0.058 | 0.225 | 0.424 | 0.518 |
| | | 2 | 0.062 | 0.209 | 0.414 | 0.532 |
| C | 16528 | 1 | * | 0.212 | 0.431 | 0.546 |
| | | 2 | * | 0.229 | 0.400 | 0.584 |
| D | 16580 | 1 | 0.061 | 0.204 | 0.385 | 0.628 |
| | | 2 | 0.063 | 0.227 | 0.377 | 0.565 |
| E | 17046 | 1 | 0.065 | 0.242 | 0.416 | 0.621 |
| | | 2 | 0.064 | 0.216 | 0.370 | 0.672 |
| F | 17076 | 1 | 0.062 | 0.200 | 0.440 | 0.606 |
| | | 2 | 0.061 | 0.215 | 0.373 | 0.552 |
| G | 17193 | 1 | 0.060 | 0.220 | 0.390 | 0.532 |
| | | 2 | 0.063 | 0.220 | 0.391 | 0.533 |
| H | 17363 | 1 | 0.060 | 0.219 | 0.381 | 0.605 |
| | | 2 | 0.065 | 0.204 | 0.392 | 0.494 |
| I | 17617 | 1 | 0.079 | 0.227 | 0.405 | 0.557 |
| | | 2 | 0.074 | 0.211 | 0.376 | 0.560 |

| | | | | |
|---|---|---|---|---|
| AVG. | 0.063 | 0.216 | 0.398 | 0.555 |
| STD. DEV. | 0.005 | 0.011 | 0.021 | 0.057 |
| % CV | 8.5 | 5.0 | 5.4 | 10.2 |

*Data point excluded because sample duplicates exceeded a % CV of 15%

Next, the Soy ELISA assay was conducted using 5% Soy Formula as the cutoff calibrator, and 1% and 10% Soy Formula as the negative and positive controls, respectively. Adulterated and unadulterated breast milk samples were analyzed using 5% Soy Formula (bold) as cutoff calibrator. The average absorbance values are presented in Table 12.

TABLE 12A
Accuracy Validation Data of Adulteration with Soy
Formula for the Veratox ® Soy ELISA Assay

| Do- | | Run 1 | | | Run 2 | | |
|---|---|---|---|---|---|---|---|
| nors | | Adulteration | | | Adulteration | | |
| ID | Number | 1% | 5% | 10% | 1% | 5% | 10% |
| A.1 | 15607 | 0.101 | 0.306 | 0.495 | 0.105 | 0.238 | 0.436 |
| A.2 | 15966 | 0.103 | 0.302 | 0.488 | 0.103 | 0.261 | 0.415 |
| B | 16226 | 0.121 | 0.275 | 0.497 | 0.099 | 0.219 | 0.383 |
| C | 16528 | 0.124 | 0.276 | 0.478 | 0.106 | 0.232 | 0.390 |
| D | 16580 | 0.119 | 0.249 | 0.509 | 0.114 | 0.238 | 0.386 |
| E | 17046 | 0.110 | 0.253 | 0.486 | 0.126 | 0.242 | 0.412 |
| F | 17076 | 0.118 | 0.285 | 0.516 | 0.131 | 0.251 | 0.417 |
| G | 17193 | 0.124 | 0.289 | 0.512 | 0.109 | 0.226 | 0.413 |
| H | 17363 | 0.110 | 0.274 | 0.462 | 0.108 | 0.208 | 0.390 |
| I | 17617 | 0.110 | 0.259 | 0.470 | 0.110 | 0.213 | 0.386 |
| | AVG. | 0.114 | 0.277 | 0.491 | 0.111 | 0.233 | 0.403 |
| | STD. DEV. | 0.008 | 0.019 | 0.018 | 0.010 | 0.017 | 0.018 |
| | % CV | 7.3 | 7.0 | 3.7 | 9.1 | 7.2 | 4.5 |

| | | |
|---|---|---|
| NEG. OD: | 0.084 | 0.087 |

| | | |
|---|---|---|
| NUMBER of CORRECT | 20/20 | 20/20 |
| RESULTS | | |
| ACCURACY | 100% | 100% |

TABLE 12B
Accuracy Validation Data of Adulteration with Soy Milk for
the Veratox ® Soy ELISA Assay

| | | Run 1 | | | Run 2 | | |
|---|---|---|---|---|---|---|---|
| | | Adulteration | | | Adulteration | | |
| Donors | | | C/O | | | C/O | |
| ID | Number | 1% | Cal* | 10% | 1% | Cal* | 10% |
| A.1 | 15607 | 0.125 | 0.260 | 0.605 | 0.126 | 0.246 | 0.622 |
| A.2 | 15966 | 0.134 | | 0.638 | 0.126 | | 0.650 |
| B | 16226 | 0.123 | | 0.589 | 0.129 | | 0.643 |
| C | 16528 | 0.121 | | 0.631 | 0.120 | | 0.676 |
| D | 16580 | 0.119 | | 0.605 | 0.121 | | 0.702 |
| E | 17046 | 0.124 | | 0.667 | 0.126 | | 0.718 |
| F | 17076 | 0.117 | | 0.670 | 0.127 | | 0.582 |
| G | 17193 | 0.115 | | 0.714 | 0.135 | | 0.631 |
| H | 17363 | 0.131 | | 0.649 | 0.139 | | 0.681 |
| I | 17617 | 0.121 | | 0.641 | 0.129 | | 0.624 |
| | AVG. | 0.123 | | 0.641 | 0.128 | | 0.653 |
| | STD. | 0.006 | | 0.037 | 0.006 | | 0.041 |
| | DEV. | | | | | | |
| | % CV | 4.8 | | 5.8 | 4.5 | | 6.3 |

*Average cutoff calibrator calculated from the individual donors and a ten donor pool

TABLE 13
Determination of 5% Soy Formula Cutoff Calibrators:

| | | |
|---|---|---|
| 10-Donor Pool | 0.246 | 0.239 |
| A.2 | 0.250 | 0.255 |
| B | 0.267 | |
| C | 0.278 | |
| D | | 0.237 |
| E | | 0.251 |
| F | | 0.249 |
| AVG. | 0.260 | 0.246 |
| STD. DEV. | 0.015 | 0.008 |
| % CV | 5.7 | 3.2 |
| NEG. OD: | 0.074 | 0.071 |
| NUMBER OF CORRECT | 20/20 | 20/20 |
| RESULTS | | |
| ACCURACY | 100% | 100% |

The overall sensitivity and specificity of identifying human breast milk adulterated with at least 10% or 1% of Soy Milk and Soy Formula were 100%.

TABLE 14
Comparison of the Sensitivity and Specificity of the Data Obtained for All
Adulterants, When Analyzing Samples in Duplicate or Singlet

| Data: Average of Duplicate wells | | | Data: Individual wells | | |
|---|---|---|---|---|---|
| | P | N | | P | N |

Total Milk Allergen kit

| | | | | | | | | | | | |
|---|---|---|---|---|---|---|---|---|---|---|---|
| Overall | P | 60 | 0 | Sensitivity: | 100% | Overall | P | 120 | 0 | Sensitivity: | 99.4% |
| Test | N | 0 | 80 | Specificity: | 100% | Test | N | 1 | 160 | Specificity: | 100% |
| Results | | | | | | Results | | | | | |
| Goat | P | 20 | 0 | Sensitivity: | 100% | Goat | P | 40 | 0 | Sensitivity: | 98% |
| Milk | N | 0 | 40 | Specificity: | 100% | Milk | N | 1 | 80 | Specificity: | 100% |
| Cow | P | 20 | 0 | Sensitivity: | 100% | Cow | P | 40 | 0 | Sensitivity: | 100% |
| Milk | N | 0 | 20 | Specificity: | 100% | Milk | N | 0 | 40 | Specificity: | 100% |
| Cow | P | 20 | 0 | Sensitivity: | 100% | Cow | P | 40 | 0 | Sensitivity: | 100% |
| Formula | N | 0 | 20 | Specificity: | 100% | Formula | N | 0 | 40 | Specificity: | 100% |

Soy Allergen kit

| | | | | | | | | | | | |
|---|---|---|---|---|---|---|---|---|---|---|---|
| Test | P | 40 | 0 | Sensitivity: | 100% | Test | P | 80 | 0 | Sensitivity: | 100% |
| Results | N | 0 | 40 | Specificity: | 100% | Results | N | 0 | 80 | Specificity: | 100% |
| Soy | P | 20 | 0 | Sensitivity: | 100% | Soy | P | 40 | 0 | Sensitivity: | 100% |
| Formula | N | 0 | 20 | Specificity: | 100% | Formula | N | 0 | 40 | Specificity: | 100% |
| Soy | P | 20 | 0 | Sensitivity: | 100% | Soy | P | 40 | 0 | Sensitivity: | 100% |
| Milk | N | 0 | 20 | Specificity: | 100% | Milk | N | 0 | 40 | Specificity: | 100% |

### Example 3

### Automated Detection of Adulterants in Pooled Human Milk

This study was performed in order to determine if the manual methodologies for the detection of cow, goat, and soy proteins in human breast milk described in the previous examples may be performed using an automated system to provide a robust and reliable method for detecting adulteration of human milk pools of ten donors while consuming an insignificant volume of human milk.

Human breast milk was provided by ten donors (15607 (A.1), 15966 (A.2), 16226 (B), 16528 (C), 16580 (D), 17046 (E), 17076 (F), 17193 (G), 17363 (H), and 17617 (I)). The non-human milk and infant formula samples used were purchased from a grocery store. The following five milk and infant formula samples were used as adulterants: Cow Milk (Hiland Vitamin D milk; Grade A, pasteurized and homogenated or Horizon Organic Vitamin D milk, ultra pasteurized and homogenated, DHA Omega-3), Goat Milk (Meyenberg Ultra Pasteurized Vitamin D milk), Cow Milk-based Formula (Similac Advance Infant Formula; Complete Nutrition), Soy Milk (8^{th} Continent Soy Milk, Original flavor), and Soy-based Formula (Similac Soy Infant Formula; Isomil).

The Veratox® Total Milk Allergen and Soy Allergen ELISA kits (Neogen Corporation) described in the previous examples were also used without the recommended extraction step. The DSX automated ELISA system (Dynex Technologies) was used to perform the ELISAs. The DSX performed the wash steps as recommended in the kit manuals. The wells were washed ten times for the Total Milk Allergen ELISA, and the wells were washed five times for the Soy Allergen ELISA. Optical densities (OD), or absorbance, were measured at 650 nm. Using the automated ELISA system, OD values were measured ten times over a period of about 16 minutes beginning at about ten minutes after the initial reading at the conclusion of each assay. OD values were recorded, and the results were determined to be positive or negative for adulteration when compared against the average of the respective cutoff calibrators.

Samples and cutoff calibrators were prepared according to the parameters in Table 15. Human milk from each of the ten donors was pooled at 100 μL each to prepare a 1 mL ten donor pooled sample. For a predefined adulteration level, e.g., 20% goat milk, in Table 1, one donor sample in the pool was appropriately adulterated, e.g., spiked with 20% goat milk, prior to adding it to the milk from the other nine unadulterated donor samples. Therefore, the overall adulterant percent in the ten donor pooled sample was only one-tenth of the claimed percent value as a result of the 10-fold dilution of the adulterated donor sample in the pool, e.g., 2% goat milk. As in the previous Example, the adulterated donor in a pool was rotated. The cutoff calibrator of the Veratox Total Milk Allergen ELISA is significantly higher than the limit of detection (LOD; OD 0.547 vs. OD 0.270). Similarly, the cutoff calibrator of the Veratox Soy Allergen ELISA is significantly higher than the LOD (OD 0.375 vs. OD 0.069).

TABLE 15
Parameters used for samples, controls, and cutoff calibrators.

| | | Adulteration Levels | |
|---|---|---|---|
| Veratox ® Kit | Adulterant | Negative | Positive |
| Total Milk Allergen | Goat Milk | 5% | 20% |
| | Cow Milk | 0.25% | 10% |
| | Cow Formula | 0.25% | 10% |
| | Cutoff Calibrator | 10% Goat Milk | |
| | Negative Control | 0% | |
| | (100% human milk) | | |
| | Positive Control | 25 ppm | |
| | (High standard) | | |
| Soy Allergen | Soy Formula | 1% | 10% |
| | Soy Milk | 1% | 10% |

| | |
|---|---|
| Cutoff Calibrator | 5% Soy Formula |
| Negative Control | 0% |
| (100% human milk) | |
| Positive Control | 25 ppm |
| (High standard) | |

The OD values generated by the automated ELISA were consistently higher than the OD values from the manual method. However, data normalized to the corresponding cutoff calibrator yielded similar OD curves regardless of whether the assay was manual or automated. It was also determined that the response was linear in that the change in OD value was proportional to the concentration of the adulterant in the sample, and the results for all adulterants analyzed using both kits were linear.

### Precision and Repeatability

In order to determine precision and reliability of the automated ELISA system, three samples were prepared for each treatment, and each sample was analyzed in singlet to produce a total of triplicate results, which is more stringent than preparing one sample and analyzing in triplicate. Precision is expressed as the standard deviation of multiple measurements of a homogeneous sample, and repeatability indicates precision within the same run or the same day. Adulteration levels were 10% goat milk for the Veratox® Total Milk Allergen assay and 5% soy formula for the Veratox® Total Soy Allergen assay. Ten donor pools were generated in which the donor sample that was adulterated was rotated.

All ten donor pools generated similar results (Tables 16A and 16B). Both the Total Milk Allergen and Soy Allergen assays demonstrated excellent precision (% CV≦10.5% within the same run) and repeatability (% CV≦15% of the two runs) using the automated ELISA system.

TABLE 16A
Precision and Repeatability of the Veratox ® Total Milk Allergen ELISA on the
DSX Automated System.

| Time | RUN 1: DONORS | | | | | | | | | | | | |
|---|---|---|---|---|---|---|---|---|---|---|---|---|---|
| (min) | D | H | B | F | I | G | A.2 | C | E | A.1 | AVG | SD | % CV |
| 10 | 0.624 | 0.642 | 0.764 | 0.686 | 0.611 | 0.618 | 0.605 | 0.658 | 0.619 | 0.626 | 0.645 | 0.048 | 7.5 |
| 12 | 0.618 | 0.637 | 0.761 | 0.679 | 0.608 | 0.615 | 0.600 | 0.652 | 0.615 | 0.608 | 0.639 | 0.049 | 7.7 |
| 14 | 0.616 | 0.635 | 0.758 | 0.680 | 0.607 | 0.615 | 0.599 | 0.651 | 0.613 | 0.604 | 0.638 | 0.049 | 7.7 |
| 16 | 0.615 | 0.633 | 0.756 | 0.676 | 0.606 | 0.614 | 0.598 | 0.650 | 0.612 | 0.603 | 0.636 | 0.048 | 7.6 |
| 18 | 0.614 | 0.633 | 0.755 | 0.673 | 0.606 | 0.614 | 0.597 | 0.649 | 0.610 | 0.619 | 0.637 | 0.047 | 7.4 |
| 20 | 0.614 | 0.632 | 0.754 | 0.675 | 0.605 | 0.614 | 0.597 | 0.649 | 0.610 | 0.600 | 0.635 | 0.048 | 7.6 |
| 21 | 0.614 | 0.632 | 0.754 | 0.674 | 0.605 | 0.613 | 0.597 | 0.648 | 0.609 | 0.609 | 0.636 | 0.048 | 7.5 |
| 23 | 0.613 | 0.632 | 0.753 | 0.672 | 0.604 | 0.613 | 0.597 | 0.648 | 0.609 | 0.600 | 0.634 | 0.048 | 7.6 |
| 25 | 0.613 | 0.631 | 0.753 | 0.674 | 0.604 | 0.612 | 0.596 | 0.647 | 0.609 | 0.609 | 0.635 | 0.048 | 7.5 |
| 26 | 0.612 | 0.631 | 0.752 | 0.674 | 0.604 | 0.612 | 0.596 | 0.648 | 0.609 | 0.598 | 0.634 | 0.048 | 7.6 |
| AVG | 0.615 | 0.634 | 0.756 | 0.676 | 0.606 | 0.614 | 0.598 | 0.650 | 0.612 | 0.608 | Over- | AVG | 0.637 |
| SD | 0.003 | 0.003 | 0.004 | 0.004 | 0.002 | 0.002 | 0.003 | 0.003 | 0.003 | 0.009 | all | SD | 0.046 |
| % CV | 0.6 | 0.5 | 0.5 | 0.6 | 0.4 | 0.3 | 0.5 | 0.5 | 0.5 | 1.5 | | % CV | 7.2 |
| Time | RUN 2: DONORS | | | | | | | | | | | | |
| (min) | G | B | E | I | F | C | D | A.1 | H | A.2 | AVG | SD | % CV |
| 10 | 0.588 | 0.547 | 0.643 | 0.569 | 0.592 | 0.649 | 0.550 | 0.521 | 0.516 | 0.572 | 0.575 | 0.045 | 7.9 |
| 12 | 0.584 | 0.544 | 0.645 | 0.571 | 0.590 | 0.646 | 0.547 | 0.519 | 0.512 | 0.574 | 0.573 | 0.046 | 8.0 |
| 14 | 0.582 | 0.543 | 0.644 | 0.569 | 0.589 | 0.643 | 0.545 | 0.517 | 0.510 | 0.569 | 0.571 | 0.046 | 8.1 |
| 15 | 0.581 | 0.541 | 0.644 | 0.567 | 0.587 | 0.641 | 0.544 | 0.517 | 0.509 | 0.569 | 0.570 | 0.046 | 8.1 |
| 17 | 0.580 | 0.544 | 0.643 | 0.566 | 0.587 | 0.641 | 0.543 | 0.516 | 0.509 | 0.569 | 0.570 | 0.046 | 8.0 |
| 19 | 0.580 | 0.544 | 0.643 | 0.565 | 0.586 | 0.640 | 0.543 | 0.576 | 0.508 | 0.567 | 0.569 | 0.046 | 8.0 |
| 21 | 0.580 | 0.544 | 0.642 | 0.564 | 0.586 | 0.640 | 0.543 | 0.516 | 0.508 | 0.566 | 0.569 | 0.046 | 8.0 |
| 22 | 0.579 | 0.544 | 0.642 | 0.563 | 0.586 | 0.639 | 0.543 | 0.516 | 0.508 | 0.568 | 0.569 | 0.045 | 8.0 |
| 24 | 0.579 | 0.544 | 0.642 | 0.563 | 0.585 | 0.639 | 0.543 | 0.516 | 0.508 | 0.567 | 0.569 | 0.045 | 8.0 |
| 26 | 0.579 | 0.543 | 0.642 | 0.563 | 0.585 | 0.639 | 0.543 | 0.516 | 0.508 | 0.567 | 0.569 | 0.045 | 8.0 |
| AVG | 0.581 | 0.544 | 0.643 | 0.566 | 0.587 | 0.642 | 0.544 | 0.517 | 0.510 | 0.569 | Over- | AVG | 0.570 |
| SD | 0.003 | 0.001 | 0.001 | 0.003 | 0.002 | 0.003 | 0.002 | 0.002 | 0.003 | 0.002 | all | SD | 0.044 |
| % CV | 0.5 | 0.3 | 0.2 | 0.5 | 0.4 | 0.5 | 0.4 | 0.3 | 0.5 | 0.4 | | % CV | 7.6 |

TABLE 16B
Precision and Repeatability of the Veratox ® Soy Allergen ELISA on the DSX
Automated System.

| Time | RUN 1: DONORS | | | | | | | | | | | | |
|---|---|---|---|---|---|---|---|---|---|---|---|---|---|
| (min) | D | H | B | F | I | G | A.2 | C | E | A.1 | AVG | SD | % CV |
| 10 | 0.390 | 0.444 | 0.391 | 0.390 | 0.377 | 0.385 | 0.329 | 0.323 | 0.317 | 0.343 | 0.369 | 0.040 | 10.8 |
| 12 | 0.391 | 0.445 | 0.391 | 0.390 | 0.378 | 0.386 | 0.330 | 0.323 | 0.318 | 0.344 | 0.370 | 0.040 | 10.8 |
| 13 | 0.391 | 0.445 | 0.392 | 0.390 | 0.378 | 0.386 | 0.331 | 0.324 | 0.319 | 0.344 | 0.370 | 0.040 | 10.7 |
| 15 | 0.392 | 0.445 | 0.392 | 0.390 | 0.378 | 0.387 | 0.331 | 0.324 | 0.319 | 0.344 | 0.370 | 0.040 | 10.8 |
| 17 | 0.392 | 0.445 | 0.393 | 0.391 | 0.379 | 0.387 | 0.331 | 0.325 | 0.319 | 0.345 | 0.371 | 0.040 | 10.7 |
| 19 | 0.392 | 0.446 | 0.393 | 0.391 | 0.379 | 0.3$7 | 0.332 | 0.325 | 0.320 | 0.345 | 0.371 | 0.040 | 10.7 |
| 20 | 0.392 | 0.445 | 0.393 | 0.391 | 0.379 | 0.387 | 0.332 | 0.325 | 0.320 | 0.345 | 0.371 | 0.039 | 10.6 |
| 22 | 0.393 | 0.446 | 0.393 | 0.391 | 0.379 | 0.387 | 0.332 | 0.326 | 0.320 | 0.346 | 0.371 | 0.040 | 10.7 |
| 24 | 0.392 | 0.446 | 0.393 | 0.391 | 0.379 | 0.387 | 0.332 | 0.326 | 0.320 | 0.345 | 0.371 | 0.040 | 10.7 |
| 26 | 0.392 | 0.445 | 0.393 | 0.391 | 0.379 | 0.387 | 0.333 | 0.326 | 0.320 | 0.346 | 0.371 | 0.039 | 10.6 |
| AVG | 0.392 | 0.445 | 0.392 | 0.391 | 0.379 | 0.387 | 0.331 | 0.325 | 0.319 | 0.345 | Overall | AVG | 0.370 |
| SD | 0.001 | 0.001 | 0.001 | 0.001 | 0.001 | 0.001 | 0.001 | 0.001 | 0.001 | 0.001 | | SD | 0.038 |
| % CV | 0.2 | 0.1 | 0.2 | 0.1 | 0.2 | 0.2 | 0.3 | 0.4 | 0.3 | 0.3 | | % CV | 10.2 |
| Time | RUN 2: DONORS | | | | | | | | | | | | |
| (min) | G | B | E | I | F | C | D | A.1 | H | A.2 | AVG | SD | % CV |
| 10 | 0.549 | 0.493 | 0.480 | 0.382 | 0.432 | 0.455 | 0.443 | 0.455 | 0.460 | 0.432 | 0.458 | 0.044 | 9.6 |
| 12 | 0.549 | 0.493 | 0.479 | 0.382 | 0.432 | 0.455 | 0.442 | 0.454 | 0.459 | 0.432 | 0.458 | 0.044 | 9.6 |
| 14 | 0.549 | 0.493 | 0.479 | 0.382 | 0.431 | 0.454 | 0.442 | 0.454 | 0.459 | 0.432 | 0.458 | 0.044 | 9.6 |
| 15 | 0.548 | 0.492 | 0.479 | 0.381 | 0.431 | 0.454 | 0.442 | 0.454 | 0.458 | 0.432 | 0.457 | 0.044 | 9.6 |
| 17 | 0.548 | 0.492 | 0.478 | 0.381 | 0.431 | 0.454 | 0.441 | 0.453 | 0.458 | 0.431 | 0.457 | 0.044 | 9.6 |
| 19 | 0.547 | 0.492 | 0.478 | 0.381 | 0.431 | 0.453 | 0.441 | 0.453 | 0.458 | 0.431 | 0.457 | 0.044 | 9.6 |
| 21 | 0.547 | 0.491 | 0.478 | 0.381 | 0.430 | 0.453 | 0.441 | 0.452 | 0.458 | 0.431 | 0.456 | 0.044 | 9.6 |
| 22 | 4.547 | 0.491 | 0.478 | 0.381 | 0.430 | 0.453 | 0.441 | 0.452 | 0.457 | 0.431 | 0.456 | 0.044 | 9.6 |
| 24 | 0.547 | 0.490 | 0.477 | 0.381 | 0.430 | 0.453 | 0.440 | 0.452 | 0.457 | 0.430 | 0.456 | 0.044 | 9.6 |
| 26 | 0.546 | 0.490 | 0.476 | 0.380 | 0.429 | 0.452 | 0.440 | 0.452 | 0.457 | 0.430 | 0.455 | 0.044 | 9.6 |
| AVG | 0.548 | 0.492 | 0.478 | 0.381 | 0.431 | 0.454 | 0.441 | 0.453 | 0.458 | 0.431 | Overall | AVG | 0.457 |
| SD | 0.001 | 0.001 | 0.001 | 0.001 | 0.001 | 0.001 | 0.001 | 0.001 | 0.001 | 0.001 | | SD | 0.042 |
| % CV | 0.2 | 0.2 | 0.2 | 0.2 | 0.2 | 0.2 | 0.2 | 0.2 | 0.2 | 0.2 | | % CV | 9.1 |

### Robustness: Sample Stability

Because proteins stored at 4° C. or at 20° C. in frost-free freezers that cycle the temperature higher and lower can be degraded and/or aggregated, the effects of the duration of storage at 4° C. and −20° C. and the number of freeze-thaw cycles had on the assays were examined. The respective cutoff calibrators (10% Goat Milk and 5% Soy Formula) and samples adulterated with 20% goat milk were used. 20% goat milk was included because it produces an OD signal closest to its cutoff calibrator, and, therefore, it is the adulteration level that is most likely to generate false negatives if storage duration or freeze-thaw cycles are to decrease the OD values of the sample. The other positive and negative adulteration levels generate ODs that are significantly higher or lower than their respective cutoff calibrators.

Samples were 3 mL volumes of human milk adulterated to the appropriate level. Each sample was mixed well and divided into three aliquots. The first aliquot was stored at 4° C., and the other two aliquots were stored at −20° C. in a frost-free freezer. One frozen aliquot was kept frozen until the day of the analysis for one freeze-thaw cycle, while the other was thawed once about halfway through the storage time and refrozen until it was thawed for analysis for two freeze-thaw cycles. Samples were stored for 5, 7 and 14 days prior to analysis. On the day of analysis, fresh 10% goat milk and 5% soy formula cutoff calibrators were prepared, and all of the other samples were compared to the OD of the fresh cutoff calibrators.

The results of the stability assays are provided in FIGS. 1 and 2. For both the Veratox® Total Milk Allergen and Soy Allergen assays, storage duration reduced OD signal, regardless of the storage temperature. Also, samples subjected to two freeze-thaw cycles also had reduced OD signals. In particular, 20% goat milk samples frozen for 14 days had an OD value that was lower than the 10% goat milk fresh cutoff calibrator, regardless of the number of freeze-thaw cycles. In order to avoid false negatives, the positive level of adulteration of goat milk was increased to 40% for the remaining studies.

### Clinical Sensitivity, Specificity and Accuracy

Assays using the Veratox® Total Milk Allergen and Soy Allergen assays were performed in order to determine the clinical sensitivity, specificity and accuracy of the assays. Sensitivity is the ability of an assay to correctly determine true positives, and specificity is the ability of an assay to correctly determine true negatives. Ten positive and ten negatives samples of each adulterant were prepared as individual samples from ten donor pools in which the adulterated donor sample was rotated. In addition, samples were blinded within each ELISA.

Tables 17A and 17B provide a summary of the performance of each assay in detecting true positives and true negatives. One 5% goat milk sample generated a false positive, and one 40% goat milk sample generated a false negative. Thus, the overall sensitivity was 98.3%, the overall specificity was 100% and the overall accuracy was 99.2% for the Veratox® Total Milk Allergen automated assay. The overall sensitivity, specificity and accuracy of the Veratox® Soy Allergen automated assay were all 100%.

TABLE 17A
Clinical Sensitivity, Specificity and Overall Accuracy of the Veratox ® Total
Milk Allergen ELISA.
Total Milk Allergen kit
Overall Results

| | | | | |
|---|---|---|---|---|
| | P | N | Sensitivity: | 98.3% |
| P | 119 | 0 | Specificity: | 100% |
| N | 1 | 120 | Accuracy: | 99.2% |

| RUN 1 | | | | | | RUN 2 | | | | | |
|---|---|---|---|---|---|---|---|---|---|---|---|
| Summary | | P | N | Sensitivity: | 96.7% | Summary | | P | N | Sensitivity: | 100% |
| Run 1 | P | 29 | 0 | Specificity: | 100% | Run 2 | P | 30 | 0 | Specificity: | 100% |
| | N | 1 | 30 | Accuracy: | 98.3% | | N | 0 | 30 | Accuracy: | 100% |
| Goat Milk | | P | N | Sensitivity: | 90% | Goat Milk | | P | N | Sensitivity: | 100% |
| | P | 9 | 0 | Specificity: | 100% | | P | 10 | 0 | Specificity: | 100% |
| | N | 1 | 10 | | | | N | 0 | 10 | | |
| Cow Milk | | P | N | Sensitivity: | 100% | Cow Milk | | P | N | Sensitivity: | 100% |
| | P | 10 | 0 | Specificity: | 100% | | P | 10 | 0 | Specificity: | 100% |
| | N | 0 | 10 | | | | N | 0 | 10 | | |
| Cow | | P | N | Sensitivity: | 100% | Cow | | P | N | Sensitivity: | 100% |
| Formula | P | 10 | 0 | Specificity: | 100% | Formula | P | 10 | 0 | Specificity: | 100% |
| | N | 0 | 10 | | | | N | 0 | 10 | | |

TABLE 17B
Clinical Sensitivity, Specificity and Overall Accuracy of the Veratox ® Soy
Allergen ELISA.
Soy Allergen kit
Overall Results

| | | | | |
|---|---|---|---|---|
| | P | N | Sensitivity: | 100% |
| P | 80 | 0 | Specificity: | 100% |
| N | 0 | 80 | Accuracy: | 100% |

| RUN 1 | | | | | | RUN 2 | | | | | |
|---|---|---|---|---|---|---|---|---|---|---|---|
| Summary | | P | N | Sensitivity: | 100% | Summary | | P | N | Sensitivity: | 100% |
| Run 1 | P | 20 | 0 | Specificity: | 100% | Run 2 | P | 20 | 0 | Specificity: | 100% |
| | N | 0 | 20 | Accuracy: | 100% | | N | 0 | 20 | Accuracy: | 100% |
| Soy | | P | N | Sensitivity: | 90% | Soy | | P | N | Sensitivity: | 100% |
| Formula | P | 10 | 0 | Specificity: | 100% | Formula | P | 10 | 0 | Specificity: | 100% |
| | N | 0 | 10 | | | | N | 0 | 10 | | |
| Soy Milk | | P | N | Sensitivity: | 100% | Soy Milk | | P | N | Sensitivity: | 100% |
| | P | 10 | 0 | Specificity: | 100% | | P | 10 | 0 | Specificity: | 100% |
| | N | 0 | 10 | | | | N | 0 | 10 | | |

### Ruggedness: Site-to-Site Comparison

In order to determine ruggedness, or the degree of reproducibility of the automated ELISAs, similarly-adulterated samples were analyzed at two different sites. Samples were prepared fresh at each facility on the day of analysis, and ten negative and ten positive samples were generated from ten-donor pools. The Total Milk Allergen and Soy Allergen assays both provided highly comparable results when the adulterated samples were analyzed by two different analysts using two different DSX automated ELISA systems. Therefore, the ruggedness of the automated assays was shown to be high as summarized in Tables 18A and 18B.

TABLE 18A
Ruggedness of the Veratox ® Total Milk Allergen ELISA.

| | Site 1 (Monrovia, CA) | | | | | Site 2 (Oklahoma City, OK) | | | | |
|---|---|---|---|---|---|---|---|---|---|---|
| Over- | | P | N | Sensitivity: | 100% | | P | N | Sensitivity: | 100% |
| all | P | 30 | 0 | Specificity: | 100% | P | 30 | 1 | Specificity: | 97% |
| Re- | N | 0 | 30 | Accuracy: | 100% | N | 0 | 29 | Accuracy: | 98.3% |
| sults | | | | | | | | | | |
| Goat | | P | N | Sensitivity: | 90% | | P | N | Sensitivity: | 100% |
| Milk | P | 10 | 0 | Specificity: | 100% | P | 10 | 1 | Specificity: | 90% |
| | N | 0 | 10 | | | N | 0 | 9 | | |
| Cow | | P | N | Sensitivity: | 100% | | P | N | Sensitivity: | 100% |
| Milk | P | 10 | 0 | Specificity: | 100% | P | 10 | 0 | Specificity: | 100% |
| | N | 0 | 10 | | | N | 0 | 10 | | |
| Cow | | P | N | Sensitivity: | 100% | | P | N | Sensitivity: | 100% |
| For- | P | 10 | 0 | Specificity: | 100% | P | 10 | 0 | Specificity: | 100% |
| mula | N | 0 | 10 | | | N | 0 | 10 | | |

TABLE 18B
Ruggedness of the Veratox ® Soy Allergen ELISA.

| | Site 1 (Monrovia, CA) | | | | | Site 2 (Oklahoma City, OK) | | | | |
|---|---|---|---|---|---|---|---|---|---|---|
| Overall | | P | N | Sensitivity: | 100% | | P | N | Sensitivity: | 100% |
| Results | P | 20 | 0 | Specificity: | 100% | P | 20 | 0 | Specificity: | 100% |
| | N | 0 | 20 | Accuracy: | 100% | N | 0 | 20 | Accuracy: | 100% |
| Soy | | P | N | Sensitivity: | 90% | | P | N | Sensitivity: | 100% |
| Formula | P | 10 | 0 | Specificity: | 100% | P | 10 | 0 | Specificity: | 100% |
| | N | 0 | 10 | | | N | 0 | 10 | | |
| Soy | | P | N | Sensitivity: | 100% | | P | N | Sensitivity: | 100% |
| Milk | P | 10 | 0 | Specificity: | 100% | P | 10 | 0 | Specificity: | 100% |
| | N | 0 | 10 | | | N | 0 | 10 | | |

In summary, Veratox® Total Milk Allergen assay was able to detect ≧0.5% cow milk, ≧1% cow formula, and ≧40% goat milk as measured against a cutoff calibrator of 10% goat milk. The Veratox® Soy Allergen assay was able to detect ≧10% soy milk and ≧10% soy formula as measured against a cutoff calibrator of 5% soy formula. Thus, both the Veratox® Total Milk Allergen and Soy Allergen ELISAs proved to be robust, precise and reproducible in detecting one adulterated donor sample pooled with nine other unadulterated donor samples the samples using the automated system. While the automated ELISAs generated higher absolute OD readings than when analyzed manually, the results were the same between the two methods of analysis when data was normalized against the cutoff value. The results were precise and repeatable using the automated system. Additionally, it was determined that samples can be assayed in singlet or triplet with similar results in terms of specificity (detection of true negatives), sensitivity (detection of true positives) and accuracy.

What is claimed is:

1. A method for testing human milk for the presence of an adulterant comprising:
(a) obtaining human milk from a donor;
(b) screening the human milk obtained in (a) for the presence of an adulterant selected from the group consisting of one or more of non-human milk and infant formula; and
(c) determining whether or not there is an adulterant present in the human milk.

2. The method of claim 1 wherein the adulterant is the milk of a non-human animal species, a plant-derived milk, or a synthetic milk.

3. The method of claim 2 wherein the adulterant is cow's milk, goat milk or soy milk.

4. The method of claim 1 wherein the adulterant is an infant formula.

5. The method of claim 4 wherein the infant formula is a cow's milk based infant formula or a soy based infant formula.

6. The method of claim 1 wherein the screening comprises ELISA, immunoblot assay, flow cytometry assay or FTIR.

7. The method of claim 6 wherein the screening comprises ELISA.

8. The method of claim 7 wherein the ELISA is performed manually or automatically.

9. The method of claim 8 wherein the ELISA is performed automatically.

10. The method of claim 6 wherein the sample is not extracted prior to screening.

11. The method of claim 1 wherein said screening is able to detect adulterants with a specificity and/or sensitivity of greater than about 80%.

12. The method of claim 1 wherein said screening is able to detect adulterants with a specificity and/or sensitivity of greater than about 85%.

13. The method of claim 1 wherein said screening is able to detect adulterants with a specificity and/or sensitivity of greater than about 90%.

14. The method of claim 1 wherein said screening is able to detect adulterants with a specificity and/or sensitivity of greater than about 95%.

15. The method of claim 1 wherein said human milk obtained in (a) is pooled with other human milk prior to the screening step in (b).

16. The method of claim 1 further comprising screening the human milk obtained in (a) for pathogens and/or drugs.

17. The method of claim 16 wherein said pathogens comprise one or more of B. cereus, HIV-1, HBV, and HCV.

18. The method of claim 16 wherein said drugs comprise one or more of amphetamine, benzodiazepine, cocaine, marijuana, methamphetamine, opiates, synthetic opioids, nicotine and their principle metabolites.
